(19) United States
(12) Patent Application Publication
(10) Pub. No.: US 2023/0158031 A1
(43) Pub. Date: May 25, 2023

(54) Treatment of Acute Respiratory Distress Syndrome and Other Disorders Involving Cytokine Storm Using BTK Inhibitors

(71) Applicant: Principia Biopharma Inc., Bridgewater, NJ (US)

(72) Inventors: Claire Langrish, Redwood City, CA (US); Dolca Thomas, San Francisco, CA (US); Stefani Wolff, San Francisco, CA (US)

(73) Assignee: Principia Biopharma Inc., Bridgewater, NJ (US)

(21) Appl. No.: 17/920,147

(22) PCT Filed: Apr. 21, 2021
(86) PCT No.: PCT/US2021/028381

§ 371 Date: Oct. 20, 2022

Related U.S. Application Data
(60) Provisional application No. 63/013,784, filed on Apr. 22, 2020.

(51) Int. Cl. A61K 31/519; A61K 45/06; A61P 37/06

(52) U.S. Cl. CPC A61K 31/519; A61K 45/06; A61P 37/06

(57) ABSTRACT

Methods of treating a disease chosen from acute respiratory distress syndrome, sepsis, sepsis induced acute lung injury, diffuse alveolar damage, macrophage activation syndrome, secondary hemophagocytic lymphohistiocytosis, cytokine release syndrome, and systemic inflammatory response syndrome in a mammal using a therapeutically effective amount of a small molecular BTK inhibitor are disclosed.

### CROSS-REFERENCE

This application claims priority to U.S. Provisional Application No. 63/013,784 filed Apr. 22, 2020, the content of which is incorporated herein by reference for all purposes.

### SUMMARY OF THE INVENTION

Disclosed herein are methods of treating a disease chosen from acute respiratory distress syndrome (ARDS), sepsis, sepsis induced acute lung injury, diffuse alveolar damage (DAD), macrophage activation syndrome (MAS), secondary hemophagocytic lymphohistiocytosis (sHIH), cytokine release syndrome (CRS), and systemic inflammatory response syndrome (SIRS) comprising administering to a mammal in need thereof a pharmaceutical composition comprising a Bruton's tyrosine kinase (BTK) inhibitor (BTKi) and a pharmaceutically acceptable carrier or excipient, wherein the BTKi is a small molecule. In some embodiments, the disease is caused by or is associated with COVID-19.

Emerging clinical data suggest that a dysregulated inflammatory immune response occurs in many severe COVID-19 patients. Severe COVID-19 patients exhibit venous thrombotic complications, complement activation, and high levels of D-dimer, a small protein fragment created when blood clots are degraded by fibrinolysis (Thachil 2020); these factors are strongly associated with high mortality rates. In addition, some COVID-19 patients suffer from a “cytokine storm” (Mehta 2020), which may contribute to the development of acute lung injury (ALI) and respiratory distress syndrome (ARDS) (Murphy 2020).

A significant increase in the migration of neutrophils to the lungs is a characteristic feature of ARDS. Multiple studies indicate a correlation between the number of neutrophils in the alveolar space and the severity of ARDS disease (Krupa 2014). In COVID-19 patients, an increase in neutrophil count and neutrophil-to-lymphocyte ratio appears to indicate higher disease severity and poor clinical prognosis (Cao 2020). Resident alveolar and recruited macrophages also appear to play important roles in the inflammatory response process that occurs in ARDS patients (Huang 2018), and growing evidence implicates excessive monocyte/macrophage activation and the associated cytokine storm with severe COVID-19 disease related complications (Zhang 2020).

Accordingly, there is a need for novel methods of treating diseases caused by or associated with COVID-19, e.g., ARDS, sepsis, sepsis induced acute lung injury, DAD, macrophage activation syndrome MAS, sHIH, CRS, and SIRS.

The enzyme BTK is a member of the Tec family non-receptor tyrosine kinases. BTK is an immunological target expressed in most hematopoietic cells, including B cells, and innate immune cells such as neutrophils, macrophages, and mast cells. The enzyme is also expressed in platelets which participate in inflammatory responses, supporting complement generation and promoting cytokine release, coagulation, and neutrophil NET formation (Busygina 2018). BTK plays a role in the development and activation of B cells and regulates immune cell functions through a variety of signaling pathways, including signaling pathways involving B cell receptors, Fc receptors, integrins, Toll-like receptor, and chemokine receptors (Rip 2018). In addition, BTK plays an important role in degranulation, migration, and retention of neutrophils in injured tissues (Herter 2018) and in monocyte/macrophage activation and differentiation processes (Rip 2018). BTK inhibition results in the modulation of various inflammatory immune cell activities such as proliferation, differentiation, and cytokine production without depleting immune cells (Rip 2018).

Without wishing to be bound by theory, BTK inhibitors may be useful in the treatment of ARDS, sepsis, sepsis induced acute lung injury, DAD, macrophage activation syndrome MAS, sHIH, CRS, and SIRS due to their potential for modulating various immune responses. BTK inhibition is protective in rodent models of ALI and ARDS, with attenuation of lung pathology, inflammation, and lung dysfunction observed in rodents dosed with BTK inhibitors. In the lung, BTKi treatment reduced alveolar macrophage and systemic neutrophil activation, and substantially diminished further monocyte and neutrophil influx. BTK inhibition also prevented the release of proinflammatory cytokines, neutrophil NET formation, and matrix metalloproteinases, which are pathogenic in acute lung injury (Florence 2018; Huang 2018; DePorto 2019). In a liver model, BTKi treatment was capable of inhibiting neutrophil activation and migration and reversing the potentially detrimental effect of neutrophil accumulation at sites of tissue inflammation and injury (Herter 2018). An anti-thrombotic effect has also been observed following BTK inhibition due to the inhibition of proinflammatory platelet mechanisms; fortuitously, BTK inhibition spares normal hemostatic platelet function (Busygina 2018).

Disclosed herein are methods of treating a disease chosen from ARDS, sepsis, sepsis induced acute lung injury, DAD, macrophage activation syndrome MAS, sHIH, CRS, and SIRS comprising administering to a mammal in need thereof a pharmaceutical composition comprising a BTK inhibitor and a pharmaceutically acceptable carrier or excipient, wherein the BTK inhibitor is a small molecule. In some embodiments of the present disclosure, the BTK inhibitor is a compound of Formula (I) or a pharmaceutically acceptable salt thereof:

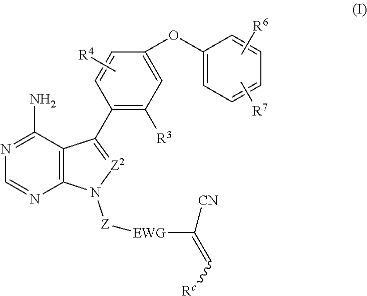

wherein:

Z² is —N— or CR², wherein R² is chosen from hydrogen and alkyl;

R³ and R⁴ are independently chosen from hydrogen, methyl, chloro, fluoro, cyclopropyl, hydroxy, methoxy, cyano, trifluoromethyl, and trifluoromethoxy;

R⁶ and R⁷ are independently chosen from hydrogen, methyl, methoxy, fluoro, chloro, trifluoromethyl, trifluoromethoxy, and cyano;

—Z-EWG- is chosen from -alkylene-NR′CO—, -alkylene-NR′SO₂—,

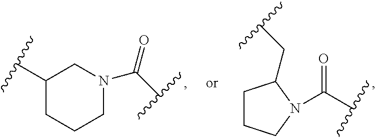 [SMILES: *C(=O)N1CCCC(*)C1 ; *CC1CCCN1C(*)=O]

or
wherein:

each of

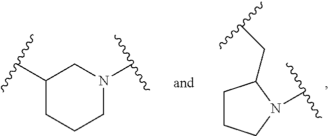 [SMILES: *C1CCCN(*)C1 ; *CC1CCCN1*]

is independently substituted with 0, 1, or 2 substituents independently chosen from alkyl, hydroxy, and halo;

the carbonyl or the sulfonyl group in -alkylene-NR′CO—, -alkylene-NR′SO₂—,

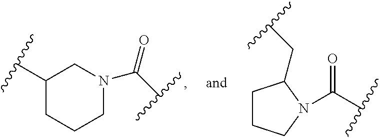 [SMILES: *C(=O)N1CCCC(*)C1 ; *CC1CCCN1C(*)=O]

and is attached to —C(CN)═CHR^{c}; and

R′ is independently chosen from hydrogen and alkyl;

R^{c} is chosen from alkyl, haloalkoxy, substituted alkyl, cycloalkyl, cycloalkylene-NR^{d}R^{e}, and cycloalkylene-alkylene-NR^{d}R^{e}; and

R^{d} and R^{e} are independently chosen from hydrogen, alkyl, cycloalkyl, and 3 to 6 membered saturated monocyclic heterocyclyls, wherein:
the heterocycles comprise one or two heteroatoms independently chosen from N, O, and S; and
the heterocycles are substituted with 0, 1, or 2 substituents independently chosen from hydroxy, alkyl, and fluoro.

Compounds of Formula (I) are disclosed in PCT/US2012/038092, which published as WO2012/158764, and PCT/US2013/058614, which published as WO2014/039899.

In some embodiments, the BTK inhibitor is a compound of Formula (I) or a pharmaceutically acceptable salt thereof, wherein Z² is —N—.

In some embodiments, the BTK inhibitor is a compound of Formula (I) or a pharmaceutically acceptable salt thereof, wherein:
Z-EWG- is

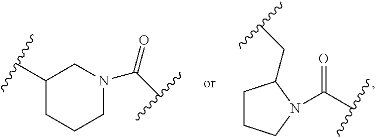 [SMILES: *C(=O)N1CCCC(*)C1 ; *CC1CCCN1C(*)=O]

wherein:

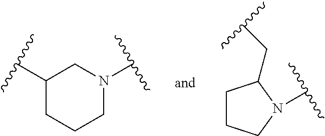 [SMILES: *C1CCCN(*)C1 ; *CC1CCCN1*]

and are independently substituted with 0, 1, or 2 substituents independently chosen from alkyl, hydroxy, and halo, and
the carbonyl group in

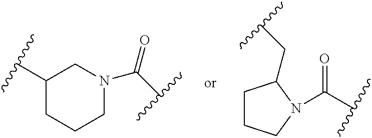 [SMILES: *C(=O)N1CCCC(*)C1 ; *CC1CCCN1C(*)=O]

is attached to —C(CN)═CHR^{c}.

In some embodiments, the BTK inhibitor is a compound of Formula (I) or a pharmaceutically acceptable salt thereof, wherein:

Z² is —N—; and

—Z-EWG- is

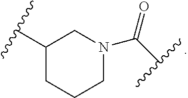 [SMILES: *C(=O)N1CCCC(*)C1]

In some embodiments, the BTK inhibitor is a compound of Formula (I) or a pharmaceutically acceptable salt thereof, wherein:

Z² is —N—; and

—Z-EWG- is

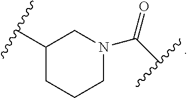 [SMILES: *C(=O)N1CCCC(*)C1]

In some embodiments, the BTK inhibitor is chosen from (E) isomer, (Z) isomer, and a mixture of (E) and (Z) isomers of any of the compounds shown in the Table 1 below, or a pharmaceutically acceptable salt of any of the foregoing:

TABLE 1

| | | Example # from |
|---|---|---|
| Compound Name | Compound Structure | WO2012/158764 |
| (R)-2-(3-(4-amino-3-(4- phenoxyphenyl)-1H-pyrazolo[3,4- d]pyrimidin-1-yl)piperidine-1- carbonyl)-3-cyclopropylacrylonitrile | 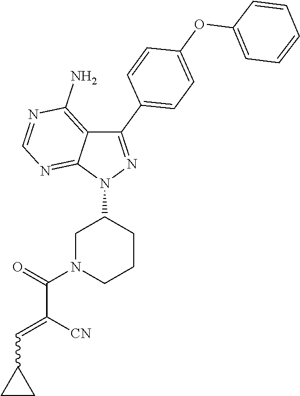 | 1 |
| 2-((R)-3-(4-amino-3-(4-(3,4- dichlorophenoxy)-3- methoxyphenyl)-1H-pyrazolo[3,4- d]pyrimidin-1-yl)piperidine-1- carbonyl)-3-cyclopropylacrylonitrile | 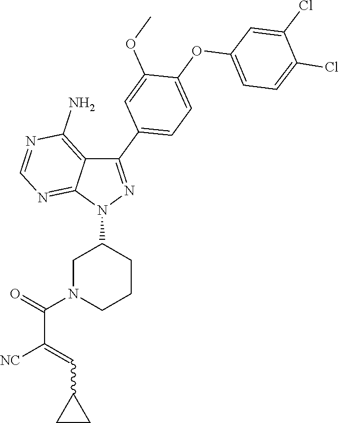 | 2 |
| (R)-2-(3-(4-amino-3-(4- phenoxyphenyl)-1H-pyrazolo[3,4- d]pyrimidin-1-yl)piperidine-1- carbonyl)-4,4-dimethylpent-2- enenitrile | 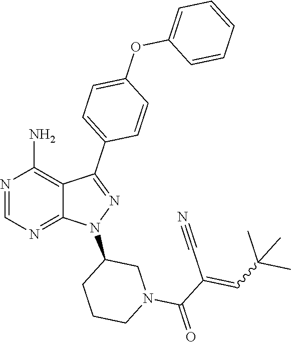 [SMILES: CC(C)(C)C=C(C#N)C(=O)N1CCC[C@@H](n2nc(-c3ccc(Oc4ccccc4)cc3)c3c(N)ncnc32)C1] | 3 |
| (R)-2-(3-(4-amino-3-(4-(3,4- dichlorophenoxy)-3- methoxyphenyl)-1H-pyrazolo-[3,4- d]pyrimidin-1-yl)piperidine-1- carbonyl)-4,4-dimethylpent-2- enenitrile | 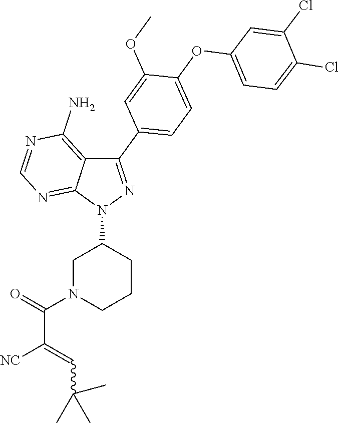 | 8 |
| (R)-2-(3-(4-amino-3-(4-(2- fluorophenoxy)phenyl)-1H- pyrazolo[3,4-d]pyrimidin-1- yl)piperidine-1-carbonyl)-3- cyclopropylacrylonitrile | 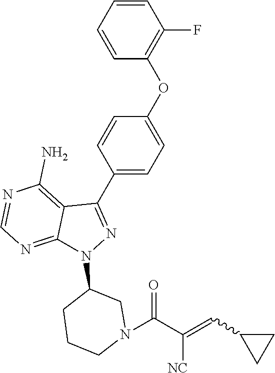 [SMILES: N#CC(=CC1CC1)C(=O)N1CCC[C@@H](n2nc(-c3ccc(Oc4ccccc4F)cc3)c3c(N)ncnc32)C1] | 16 |
| (R)-2-(3-(4-amino-3-(4-(3- fluorophenoxy)phenyl)-1H- pyrazolo[3,4-d]pyrimidin-1- yl)piperidine-1-carbonyl)-3- cyclopropylacrylonitrile | 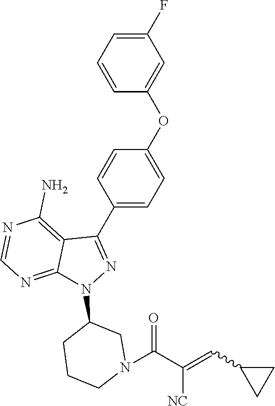 [SMILES: N#CC(=CC1CC1)C(=O)N1CCC[C@@H](n2nc(-c3ccc(Oc4cccc(F)c4)cc3)c3c(N)ncnc32)C1] | 17 |
| (R)-2-(3-(4-amino-3-(4-(2,3- difluorophenoxy)phenyl)-1H- pyrazolo[3,4-d]pyrimidin-1- yl)piperidine-1-carbonyl)-3- cyclopropylacrylonitrile | 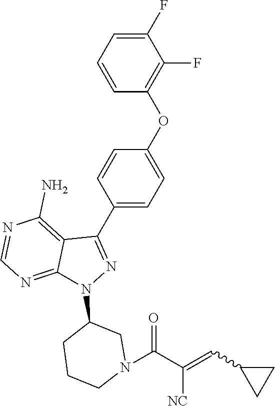 [SMILES: N#CC(=CC1CC1)C(=O)N1CCC[C@@H](n2nc(-c3ccc(Oc4cccc(F)c4F)cc3)c3c(N)ncnc32)C1] | 18 |
| (R)-2-(3-(4-amino-3-(4-(2,6- difluorophenoxy)phenyl)-1H- pyrazolo[3,4-d]pyrimidin-1- yl)piperidine-1-carbonyl)-3- cyclopropylacrylonitrile | 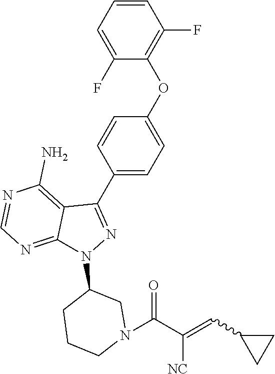 [SMILES: N#CC(=CC1CC1)C(=O)N1CCC[C@@H](n2nc(-c3ccc(Oc4c(F)cccc4F)cc3)c3c(N)ncnc32)C1] | 19 |
| (R)-2-(3-(4-amino-3-(4-(3,5- difluorophenoxy)phenyl)-1H- pyrazolo[3,4-d]pyrimidin-1- yl)piperidine-1-carbonyl)-3- cyclopropylacrylonitrile | 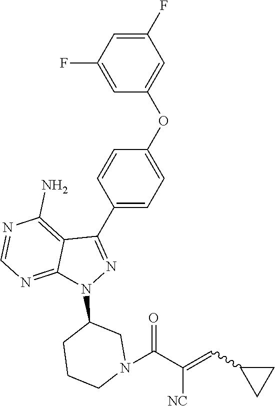 [SMILES: N#CC(=CC1CC1)C(=O)N1CCC[C@@H](n2nc(-c3ccc(Oc4cc(F)cc(F)c4)cc3)c3c(N)ncnc32)C1] | 20 |
| (R)-2-(3-(4-amino-3-(4-(2,5- difluorophenoxy)phenyl)-1H- pyrazolo[3,4-d]pyrimidin-1- yl)piperidine-1-carbonyl)-3- cyclopropylacrylonitrile | 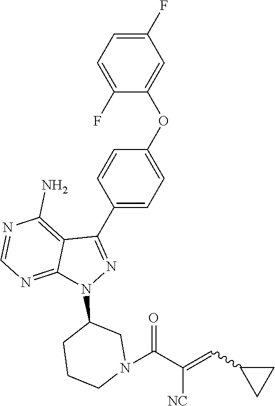 [SMILES: N#CC(=CC1CC1)C(=O)N1CCC[C@@H](n2nc(-c3ccc(Oc4cc(F)ccc4F)cc3)c3c(N)ncnc32)C1] | 21 |
| (R)-2-(3-(4-amino-3-(2-fluoro-4- (phenoxy)phenyl)-1H-pyrazolo[3,4- d]pyrimidin-1-yl)piperidine-1- carbonyl)-3-cyclopropylacrylonitrile | 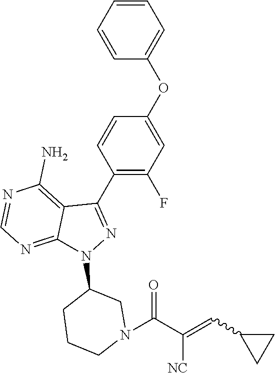 [SMILES: N#CC(=CC1CC1)C(=O)N1CCC[C@@H](n2nc(-c3ccc(Oc4ccccc4)cc3F)c3c(N)ncnc32)C1] | 22 |
| (R)-2-(3-(4-amino-5-(4- phenoxyphenyl)-7H-pyrrolo[2,3- d]pyrimidin-7-yl)piperidine-1- carbonyl)-3-cyclopropylacrylonitrile | 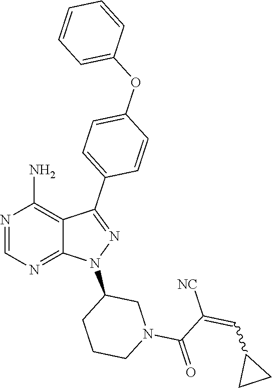 [SMILES: N#CC(=CC1CC1)C(=O)N1CCC[C@@H](n2nc(-c3ccc(Oc4ccccc4)cc3)c3c(N)ncnc32)C1] | 27 |
| (R)-2-(3-(4-amino-3-(4-(2,3- difluorophenoxy)-2-fluorophenyl)- 1H-pyrazolo[3,4-d]pyrimidin-1- yl)piperidine-1-carbonyl)-4- methylpent-2-enenitrile | 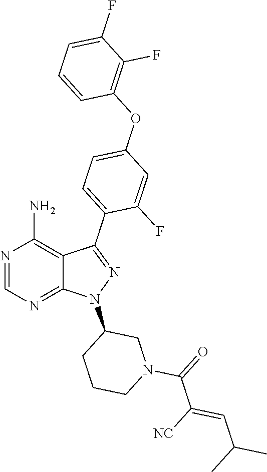 [SMILES: CC(C)/C=C(\C#N)C(=O)N1CCC[C@@H](n2nc(-c3ccc(Oc4cccc(F)c4F)cc3F)c3c(N)ncnc32)C1] | 32 |

In some embodiments, the BTK inhibitor is a compound of Formula (I) or a pharmaceutically acceptable salt thereof, wherein:

Z² is —N—; and

—Z-EWG- is

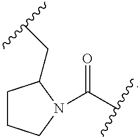 [SMILES: *CC1CCCN1C(*)=O]

In some embodiments, the BTK inhibitor is a compound of Formula (I) or a pharmaceutically acceptable salt thereof, wherein Z² is —N—; and

—Z-EWG- is

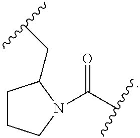 [SMILES: *CC1CCCN1C(*)=O]

In some embodiments, the BTK inhibitor is chosen from (E) isomer, (Z) isomer, and a mixture of (E) and (Z) isomers any of the compounds shown in the Table 2 below, or a pharmaceutically acceptable salt of any of the foregoing:

TABLE 2

| | | Example # from |
|---|---|---|
| Compound Name | Compound Structure | WO2012/158764 |
| 2-(2-((4-amino-3-(4-phenoxyphenyl)- 1H-pyrazolo[3,4-d]pyrimidin-1- yl)methyl)pyrrolidine-1-carbonyl)-4,4- dimethylpent-2-enenitrile | 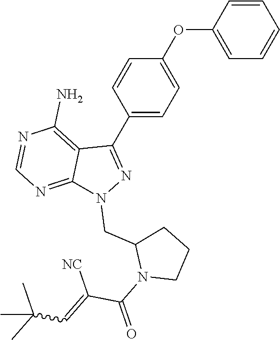 [SMILES: CC(C)(C)C=C(C#N)C(=O)N1CCCC1Cn1nc(-c2ccc(Oc3ccccc3)cc2)c2c(N)ncnc21] | 4 |
| (R)-2-(2-((4-amino-3-(4-(3- fluorophenoxy)phenyl)-1H- pyrazolo[3,4-d]pyrimidin-1- yl)methyl)pyrrolidine-1-carbonyl)-3- cyclopropylacrylonitrile | 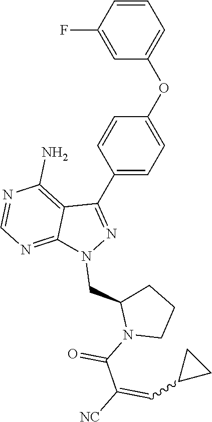 [SMILES: N#CC(=CC1CC1)C(=O)N1CCC[C@@H]1Cn1nc(-c2ccc(Oc3cccc(F)c3)cc2)c2c(N)ncnc21] | 11 |
| (R)-2-(2-((4-amino-3-(4-(2,6- difluorophenoxy)phenyl)-1H- pyrazolo[3,4-d]pyrimidin-1- yl)methyl)pyrrolidine-1-carbonyl)-3- cyclopropylacrylonitrile | 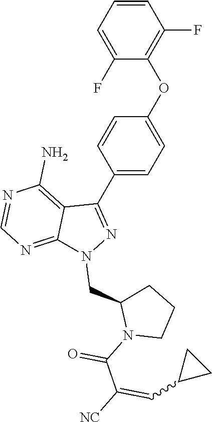 [SMILES: N#CC(=CC1CC1)C(=O)N1CCC[C@@H]1Cn1nc(-c2ccc(Oc3c(F)cccc3F)cc2)c2c(N)ncnc21] | 12 |
| (R)-2-(2-((4-amino-3-(2-fluoro-4- phenoxyphenyl)-1H-pyrazolo[3,4- d]pyrimidin-1-yl)methyl)pyrrolidine-1- carbonyl)-3-cyclopropylacrylonitrile | 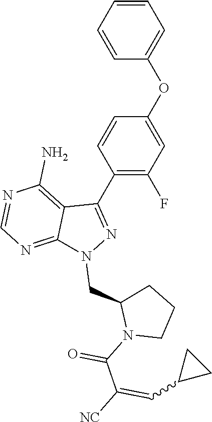 [SMILES: N#CC(=CC1CC1)C(=O)N1CCC[C@@H]1Cn1nc(-c2ccc(Oc3ccccc3)cc2F)c2c(N)ncnc21] | 13 |
| (R)-2-(2-((4-amino-3-(4-(2,3- difluorophenoxy)phenyl)-1H- pyrazolo[3,4-d]pyrimidin-1- yl)methyl)pyrrolidine-1-carbonyl)-3- cyclopropylacrylonitrile | 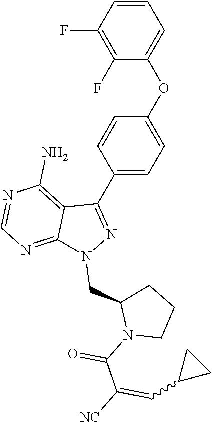 [SMILES: N#CC(=CC1CC1)C(=O)N1CCC[C@@H]1Cn1nc(-c2ccc(Oc3cccc(F)c3F)cc2)c2c(N)ncnc21] | 14 |
| (R)-2-(2-((4-amino-3-(4-(3,5- difluorophenoxy)phenyl)-1H- pyrazolo[3,4-d]pyrimidin-1- yl)methyl)pyrrolidine-1-carbonyl)-3- cyclopropylacrylonitrile | 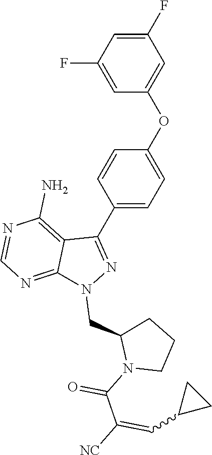 [SMILES: N#CC(=CC1CC1)C(=O)N1CCC[C@@H]1Cn1nc(-c2ccc(Oc3cc(F)cc(F)c3)cc2)c2c(N)ncnc21] | 15 |
| (R)-2-(2-((4-amino-3-(4-(2- fluorophenoxy)phenyl)-1H- pyrazolo[3,4-d]pyrimidin-1- yl)methyl)pyrrolidine-1-carbonyl)-3- cyclopropylacrylonitrile | 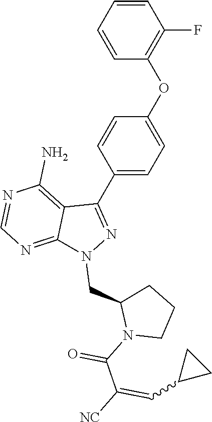 [SMILES: N#CC(=CC1CC1)C(=O)N1CCC[C@@H]1Cn1nc(-c2ccc(Oc3ccccc3F)cc2)c2c(N)ncnc21] | 23 |
| (S)-2-{2-[4-amino-5-(4-phenoxy- phenyl)-pyrrolo[2,3-d]pyrimidin-7- ylmethyl]-pyrrolidine-1-carbonyl}-3- cyclopropyl-acrylonitrile | 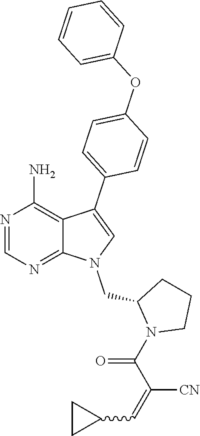 [SMILES: N#CC(=CC1CC1)C(=O)N1CCC[C@H]1Cn1cc(-c2ccc(Oc3ccccc3)cc2)c2c(N)ncnc21] | 28 |
| (S)-2-(2-((4-amino-6-methyl-5-(4- phenoxyphenyl)-7H-pyrrolo[2,3- d]pyrimidin-7-yl)methyl)pyrrolidine-1- carbonyl)-3-cyclopropylacrylonitrile | 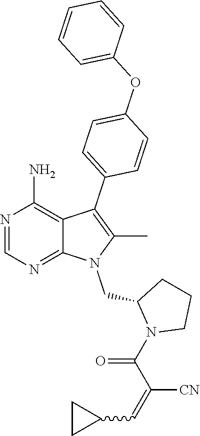 [SMILES: Cc1c(-c2ccc(Oc3ccccc3)cc2)c2c(N)ncnc2n1C[C@@H]1CCCN1C(=O)C(C#N)=CC1CC1] | 29 |
| (S)-2-(2-((4-amino-6-methyl-5-(4- phenoxyphenyl)-7H-pyrrolo[2,3- d]pyrimidin-7-yl)methyl)pyrrolidine-1- carbonyl)-4-(dimethylamino)-4- methylpent-2-enenitrile | 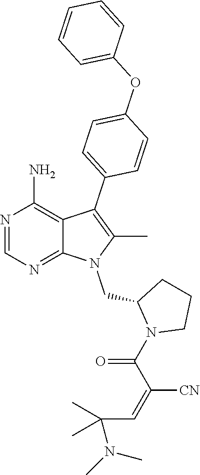 [SMILES: Cc1c(-c2ccc(Oc3ccccc3)cc2)c2c(N)ncnc2n1C[C@@H]1CCCN1C(=O)/C(C#N)=C\C(C)(C)N(C)C] | 30 |
| (S)-2-(2-((4-amino-3-(2-fluoro-4- phenoxyphenyl)-1H-pyrazolo[3,4- d]pyrimidin-1-yl)methyl)pyrrolidine-1- carbonyl)-3-cyclopropylacrylonitrile | 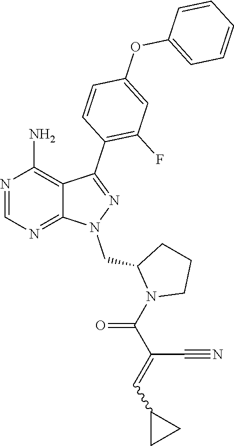 [SMILES: N#CC(=CC1CC1)C(=O)N1CCC[C@H]1Cn1nc(-c2ccc(Oc3ccccc3)cc2F)c2c(N)ncnc21] | 34 |
| (R)-2-(2-((4-amino-3-(4-(2,3- difluorophenoxy)-2-fluorophenyl)-1H- pyrazolo[3,4-d]pyrimidin-1- yl)methyl)pyrrolidine-1-carbonyl)-3- cyclopropylacrylonitrile | 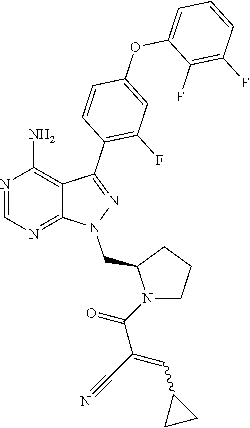 [SMILES: N#CC(=CC1CC1)C(=O)N1CCC[C@@H]1Cn1nc(-c2ccc(Oc3cccc(F)c3F)cc2F)c2c(N)ncnc21] | 37 |
| (S)-2-(2-((4-amino-3-(4-(2,3- difluorophenoxy)-2-fluorophenyl)-1H- pyrazolo[3,4-d]pyrimidin-1- yl)methyl)pyrrolidine-1-carbonyl)-3- cyclopropylacrylonitrile | 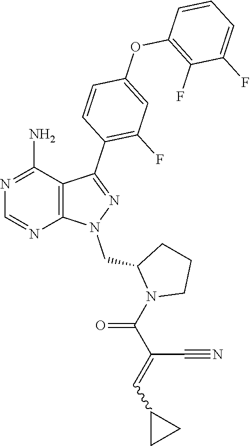 [SMILES: N#CC(=CC1CC1)C(=O)N1CCC[C@H]1Cn1nc(-c2ccc(Oc3cccc(F)c3F)cc2F)c2c(N)ncnc21] | 38 |
| 2-((S)-2-((4-amino-3-(2-fluoro-4- phenoxyphenyl)-1H-pyrazolo[3,4- d]pyrimidin-1-yl)methyl)pyrrolidine-1- carbonyl)-4-(ethylamino)-4-methylpent- 2-enenitrile | 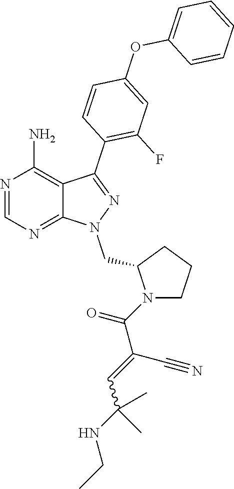 [SMILES: CCNC(C)(C)C=C(C#N)C(=O)N1CCC[C@H]1Cn1nc(-c2ccc(Oc3ccccc3)cc2F)c2c(N)ncnc21] | 40 |
| 2-((R)-2-((4-amino-3-(2-fluoro-4- phenoxyphenyl)-1H-pyrazolo[3,4- d]pyrimidin-1-yl)methyl)pyrrolidine-1- carbonyl)-4-(ethylamino)-4-methylpent- 2-enenitrile | 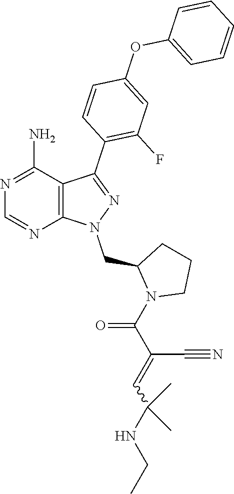 | 41 |
| 2-((S)-2-((4-amino-3-(2-fluoro-4- phenoxyphenyl)-1H-pyrazolo[3,4- d]pyrimidin-1-yl)methyl)pyrrolidine-1- carbonyl)-4-(cyclopropylamino)-4- methylpent-2-enenitrile | 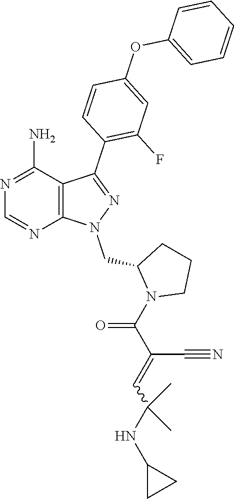 | 42 |
| 2-((S)-2-((4-amino-3-(2-fluoro-4- phenoxyphenyl)-1H-pyrazolo[3,4- d]pyrimidin-1-yl)methyl)pyrrolidine-1- carbonyl)-4-(2-methoxyethylamino)-4- methylpent-2-enenitrile | 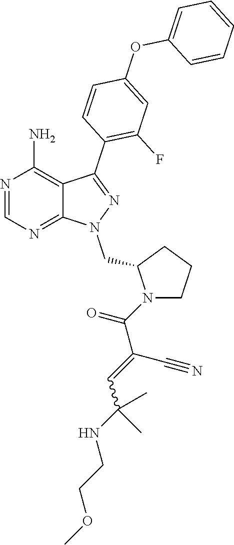 [SMILES: COCCNC(C)(C)C=C(C#N)C(=O)N1CCC[C@H]1Cn1nc(-c2ccc(Oc3ccccc3)cc2F)c2c(N)ncnc21] | 43 |
| (R)-2-(2-((4-amino-3-(2-fluoro-4- phenoxyphenyl)-1H-pyrazolo[3,4- d]pyrimidin-1-yl)methyl)pyrrolidine-1- carbonyl)-3-(l- aminocyclopropyl)acrylonitrile | 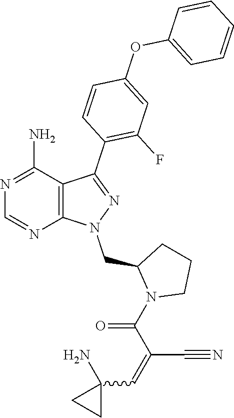 [SMILES: N#CC(=CC1(N)CC1)C(=O)N1CCC[C@@H]1Cn1nc(-c2ccc(Oc3ccccc3)cc2F)c2c(N)ncnc21] | 45 |
| 2-[(2S)-2-[[4-amino-3-(2-fluoro-4- phenoxy-phenyl)pyrazolo[3,4- d]pyrimidin-1-yl]methyl]pyrrolidine-1- carbonyl]-4-methyl-4-morpholino-pent- 2-enenitrile | 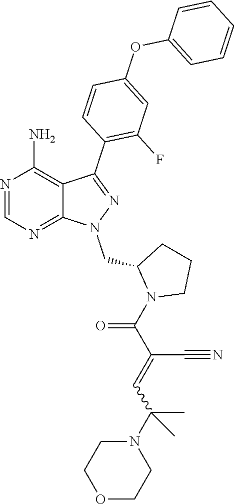 [SMILES: CC(C)(C=C(C#N)C(=O)N1CCC[C@H]1Cn1nc(-c2ccc(Oc3ccccc3)cc2F)c2c(N)ncnc21)N1CCOCC1] | 46 |
| 2-[(2R)-2-[[4-amino-3-(2-fluoro-4- phenoxy-phenyl)pyrazolo[3,4- d]pyrimidin-1-yl]methyl]pyrrolidine-1- carbonyl]-4-methyl-4-morpholino-pent- 2-enenitrile | 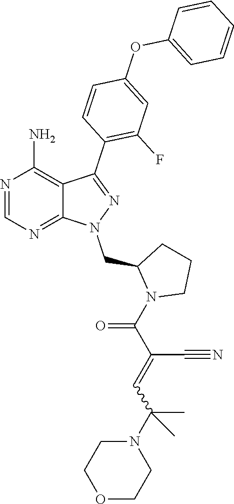 [SMILES: CC(C)(C=C(C#N)C(=O)N1CCC[C@@H]1Cn1nc(-c2ccc(Oc3ccccc3)cc2F)c2c(N)ncnc21)N1CCOCC1] | 47 |
| 2-[(2R)-2-[[4-amino-3-(2-fluoro-4- phenoxy-phenyl)pyrazolo[3,4- d]pyrimidin-1-yl]methyl]pyrrolidine-1- carbonyl]-4-methyl-4-(1-piperidyl)pent- 2-enenitrile | 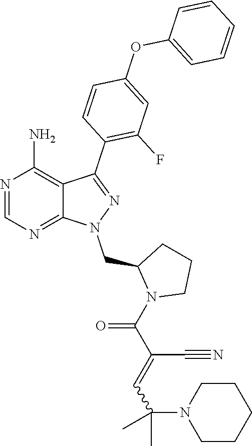 [SMILES: CC(C)(C=C(C#N)C(=O)N1CCC[C@@H]1Cn1nc(-c2ccc(Oc3ccccc3)cc2F)c2c(N)ncnc21)N1CCCCC1] | 48 |
| 2-[(2S)-2-[[4-amino-3-(2-fluoro-4- phenoxy-phenyl)pyrazolo[3,4- d]pyrimidin-1-yl]methyl]pyrrolidine-1- carbonyl]-4-methyl-4-(1-piperidyl)pent- 2-enenitrile | 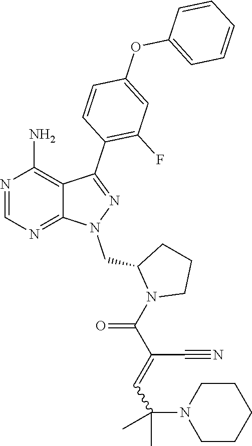 [SMILES: CC(C)(C=C(C#N)C(=O)N1CCC[C@H]1Cn1nc(-c2ccc(Oc3ccccc3)cc2F)c2c(N)ncnc21)N1CCCCC1] | 49 |
| 2-[(2S)-2-[[4-amino-3-(2-fluoro-4- phenoxy-phenyl)pyrazolo[3,4- d]pyrimidin-1-yl]methyl]-pyrrolidine-1- carbonyl]-3-(3-methyloxetan-3-yl)prop- 2-enenitrile | 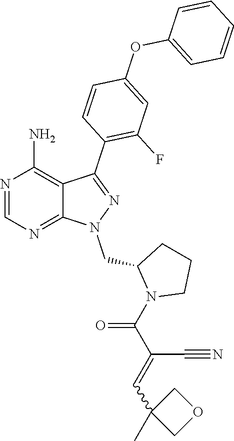 [SMILES: CC1(C=C(C#N)C(=O)N2CCC[C@H]2Cn2nc(-c3ccc(Oc4ccccc4)cc3F)c3c(N)ncnc32)COC1] | 54 |

In some embodiments, the BTK inhibitor is a compound of Formula (I) or a pharmaceutically acceptable salt thereof, wherein:

Z² is —N—;

—Z-EWG- is

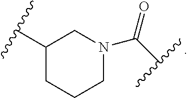 [SMILES: *C(=O)N1CCCC(*)C1]

wherein:

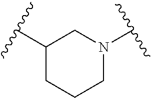 [SMILES: *C1CCCN(*)C1]

is substituted with 0, 1, or 2 substituents independently chosen from alkyl, hydroxy, and halo; and
the carbonyl group in

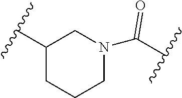 [SMILES: *C(=O)N1CCCC(*)C1]

is attached to —C(CN)═CHR^{c}; and

R^{c} is alkyl.

In some embodiments, the BTK inhibitor is a compound of Formula (I) or a pharmaceutically acceptable salt thereof, wherein:

Z² is —N—;

—Z-EWG- is

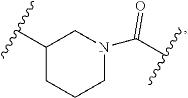 [SMILES: *C(=O)N1CCCC(*)C1]

wherein:

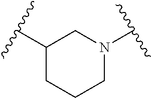 [SMILES: *C1CCCN(*)C1]

is substituted with 0, 1, or 2 substituents independently chosen from alkyl, hydroxy, and halo; and
the carbonyl group in

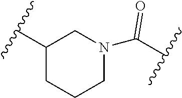 [SMILES: *C(=O)N1CCCC(*)C1]

is attached to —C(CN)═CHR^{c}; and

R^{c} is t-butyl.

In some embodiments of the disclosure, the BTK inhibitor is chosen from (E) isomer, (Z) isomer, and a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile (Compound (IA)), or a pharmaceutically acceptable salt of any of the foregoing. A non-fluorinated analog of Compound (IA) is disclosed in Example 3 of WO 2012/158764. Compound (IA) has the following structure:

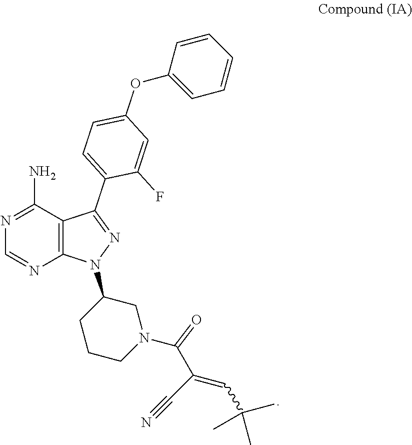 [SMILES: CC(C)(C)C=C(C#N)C(=O)N1CCC[C@@H](n2nc(-c3ccc(Oc4ccccc4)cc3F)c3c(N)ncnc32)C1]

In some embodiments, the BTK inhibitor is a substantially pure (E) or (Z) isomer of (R)-2-(3-(4-amino-3-(2-fluoro-4-phenoxyphenyl)-H-pyrazolo[3,4-d]pyrimidin-1-yl)piperidine-1-carbonyl)-4,4-dimethylpent-2-enenitrile (Compound (IA)), or a pharmaceutically acceptable salt thereof.

In some embodiments, the BTK inhibitor is a compound of Formula (I) or a pharmaceutically acceptable salt thereof, wherein

Z² is —N—;

—Z-EWG- is

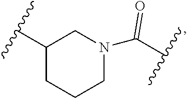 [SMILES: *C(=O)N1CCCC(*)C1]

wherein:

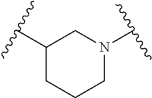 [SMILES: *C1CCCN(*)C1]

is substituted with 0, 1, or 2 substituents independently chosen from alkyl, hydroxy, and halo; and
the carbonyl group in

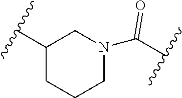 [SMILES: *C(=O)N1CCCC(*)C1]

is attached to —C(CN)═CHR^{c}; and

R^{c} is substituted alkyl.

In some embodiments, the BTK inhibitor is a compound of Formula (I) or a pharmaceutically acceptable salt thereof, wherein:

Z² is —N—;

—Z-EWG- is

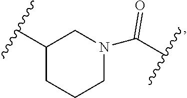 [SMILES: *C(=O)N1CCCC(*)C1]

wherein:

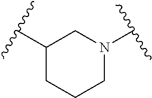 [SMILES: *C1CCCN(*)C1]

is substituted with 0, 1, or 2 substituents independently chosen from alkyl, hydroxy, and halo; and
the carbonyl group in

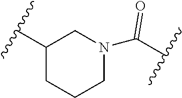 [SMILES: *C(=O)N1CCCC(*)C1]

is attached to —C(CN)═CHR^{c}; and

R^{c} is —C(CH₃)₂-(4-R⁸-piperazin-1-yl), wherein:
R⁸ is chosen from hydrogen, alkyl, alkoxyalkyl, haloalkyl, alkylsulfonyl, alkoxycarbonyl, acyl, and oxetan-3-yl; and
the piperazinyl ring is additionally optionally substituted with one or two independently chosen alkyl.

In some embodiments, the BTK inhibitor is a compound of Formula (I) or a pharmaceutically acceptable salt thereof, wherein:

Z² is —N—;

—Z-EWG- is

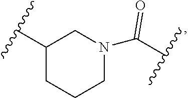 [SMILES: *C(=O)N1CCCC(*)C1]

wherein:

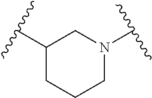 [SMILES: *C1CCCN(*)C1]

is substituted with 0, 1, or 2 substituents independently chosen from alkyl, hydroxy, and halo;
the carbonyl group in

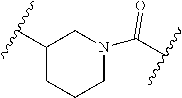 [SMILES: *C(=O)N1CCCC(*)C1]

is attached to —C(CN)═CHR^{c}; and

R^{c} is —C(CH₃)₂-(4-R⁸-piperazin-1-yl), wherein:
R⁸ is chosen from hydrogen, alkyl, alkoxyalkyl, haloalkyl, alkylsulfonyl, alkoxycarbonyl, acyl, and oxetan-3-yl; and
the piperazinyl ring is additionally optionally substituted with one or two independently chosen alkyl.

In some embodiments, the BTK inhibitor is chosen from (E) isomer, (Z) isomer, and a mixture of (E) and (Z) isomers any of the compounds shown in the Table 3 below, or a pharmaceutically acceptable salt of any of the foregoing

TABLE 3

| | | Example # from |
|---|---|---|
| Compound Name | Compound Structure | WO2014/039899 |
| 2-[[(3R)-3-[4-amino-3-(2- fluoro-4-phenoxyphenyl)-1H- pyrazolo[3,4-d]-pyrimidin-1- yl]-piperidin-1-yl]carbonyl]- 4-methyl-4-(4- methylpiperazin-1-yl)pent-2- enenitrile | 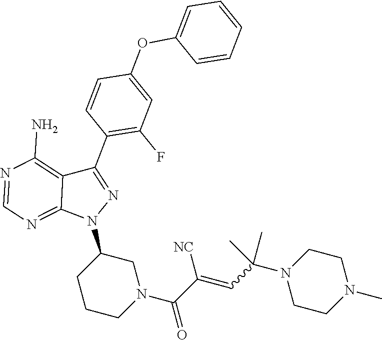 [SMILES: CN1CCN(C(C)(C)C=C(C#N)C(=O)N2CCC[C@@H](n3nc(-c4ccc(Oc5ccccc5)cc4F)c4c(N)ncnc43)C2)CC1] | 4 |
| 2-((R)-3-(4-amino-3-(2- fluoro-4-phenoxyphenyl)-1H- pyrazolo[3,4-d]pyrimidin-1- yl)piperidine-1-carbonyl)-4- methyl-4-(piperazin-1- yl)pent-2-enenitrile | 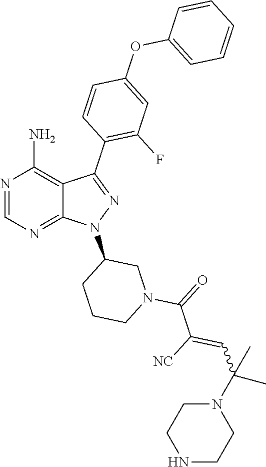 [SMILES: CC(C)(C=C(C#N)C(=O)N1CCC[C@@H](n2nc(-c3ccc(Oc4ccccc4)cc3F)c3c(N)ncnc32)C1)N1CCNCC1] | 6 |
| 2-[(3R)-3-[4-amino-3-(2- fluoro-4-phenoxy- phenyl)pyrazolo[3,4-d]- pyrimidin-1-yl]piperidine-1- carbonyl]-4-(4-ethylpiperazin- 1-yl)-4-methyl-pent-2- enenitrile | 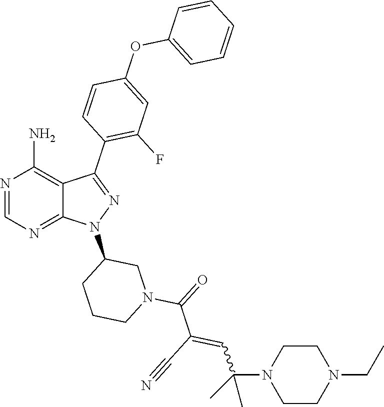 [SMILES: CCN1CCN(C(C)(C)C=C(C#N)C(=O)N2CCC[C@@H](n3nc(-c4ccc(Oc5ccccc5)cc4F)c4c(N)ncnc43)C2)CC1] | 20 |
| (R)-2-(3-(4-amino-3-(2- fluoro-4-phenoxyphenyl)-1H- pyrazolo[3,4-d]pyrimidin-1- yl)piperidine-1-carbonyl)-4- (4-isopropylpiperazin-1-yl)-4- methylpent-2-enenitrile | 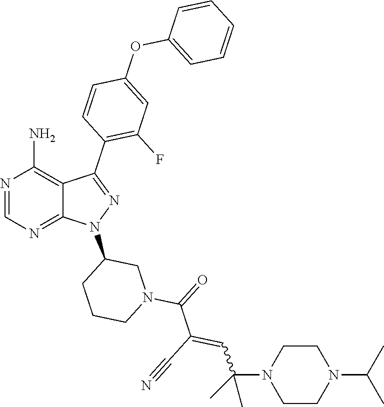 [SMILES: CC(C)N1CCN(C(C)(C)C=C(C#N)C(=O)N2CCC[C@@H](n3nc(-c4ccc(Oc5ccccc5)cc4F)c4c(N)ncnc43)C2)CC1] | 21 |
| (R)-2-(3-(4-amino-3-(2- fluoro-4-phenoxyphenyl)-1H- pyrazolo[3,4-d]pyrimidin-1- yl)piperidine-1-carbonyl)-4- (4-(tert-butyl)piperazin-1-yl)- 4-methylpent-2-enenitrile | 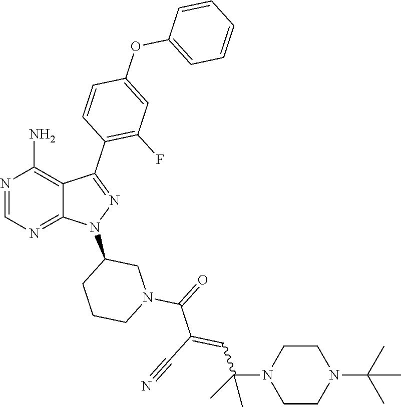 [SMILES: CC(C)(C)N1CCN(C(C)(C)C=C(C#N)C(=O)N2CCC[C@@H](n3nc(-c4ccc(Oc5ccccc5)cc4F)c4c(N)ncnc43)C2)CC1] | 22 |
| (R)-2-(3-(4-amino-3-(2- fluoro-4-phenoxyphenyl)-1H- pyrazolo[3,4-d]pyrimidin-1- yl)piperidine-1-carbonyl)-4- (4-(2-methoxyethyl)piperazin- l-yl)-4-methylpent-2- enenitrile | 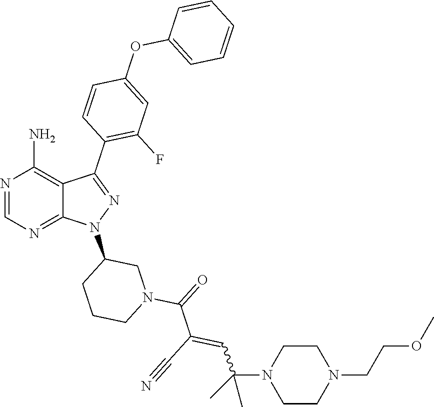 [SMILES: COCCN1CCN(C(C)(C)C=C(C#N)C(=O)N2CCC[C@@H](n3nc(-c4ccc(Oc5ccccc5)cc4F)c4c(N)ncnc43)C2)CC1] | 23 |
| (R)-2-(3-(4-amino-3-(2- fluoro-4-phenoxyphenyl)-1H- pyrazolo[3,4-d]pyrimidin-1- yl)piperidine-1-carbonyl)-4- methyl-4-(4- (methylsulfonyl)piperazin-1- yl)pent-2-enenitrile | 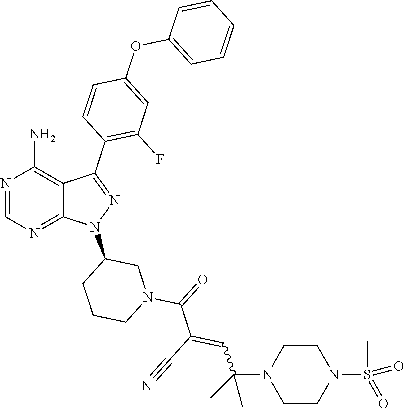 [SMILES: CC(C)(C=C(C#N)C(=O)N1CCC[C@@H](n2nc(-c3ccc(Oc4ccccc4)cc3F)c3c(N)ncnc32)C1)N1CCN(S(C)(=O)=O)CC1] | 25 |
| (R)-2-(3-(4-amino-3-(2- fluoro-4-phenoxyphenyl)-1H- pyrazolo[3,4-d]pyrimidin-1- yl)piperidine-1-carbonyl)-4- methyl-4-(4-(2,2,2- trifluoroethyl)piperazin-1- yl)pent-2-enenitrile | 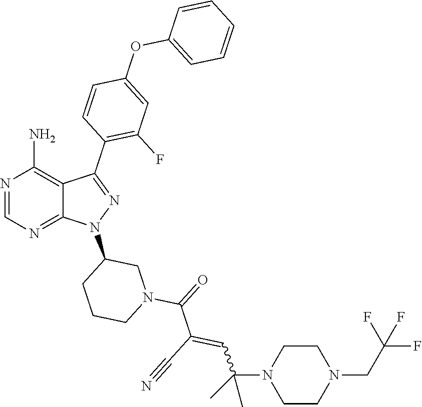 [SMILES: CC(C)(C=C(C#N)C(=O)N1CCC[C@@H](n2nc(-c3ccc(Oc4ccccc4)cc3F)c3c(N)ncnc32)C1)N1CCN(CC(F)(F)F)CC1] | 26 |
| 2-((R)-3-(4-amino-3-(2- fluoro-4-phenoxyphenyl)-1H- pyrazolo[3,4-d]pyrimidin-1- yl)piperidine-1-carbonyl)-4- methyl-4-((3S,5R)-3,4,5- trimethylpiperazin-1-yl)pent- 2-enenitrile | 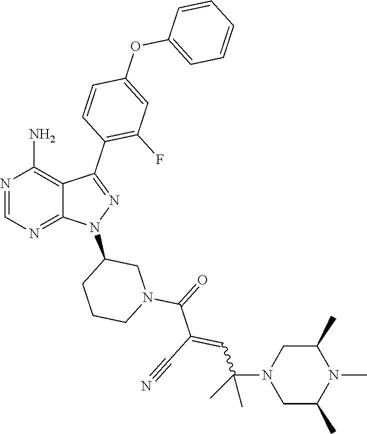 [SMILES: C[C@@H]1CN(C(C)(C)C=C(C#N)C(=O)N2CCC[C@@H](n3nc(-c4ccc(Oc5ccccc5)cc4F)c4c(N)ncnc43)C2)C[C@H](C)N1C] | 27 |
| 2-((R)-3-(4-amino-3-(2- fluoro-4-phenoxyphenyl)-1H- pyrazolo[3,4-d]pyrimidin-1- yl)piperidine-1-carbonyl)-4- ((3S,5R)-3,5- dimethylpiperazin-1-yl)-4- methylpent-2-enenitrile | 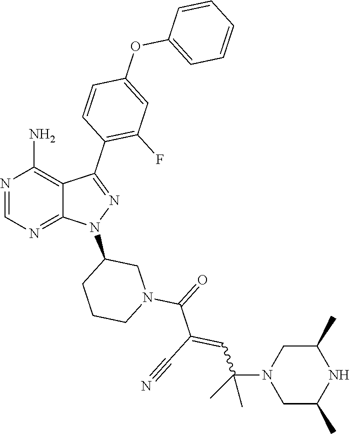 [SMILES: C[C@@H]1CN(C(C)(C)C=C(C#N)C(=O)N2CCC[C@@H](n3nc(-c4ccc(Oc5ccccc5)cc4F)c4c(N)ncnc43)C2)C[C@H](C)N1] | 28 |
| (R)-2-(3-(4-amino-3-(2- fluoro-4-phenoxyphenyl)-1H- pyrazolo[3,4-d]pyrimidin-1- yl)piperidine-1-carbonyl)-4- methyl-4-(4-(oxetan-3- yl)piperazin-1-yl)pent-2- enenitrile | 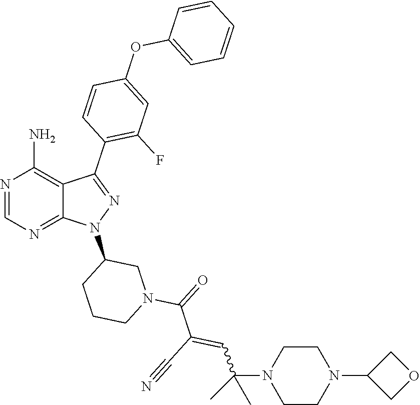 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1] | 31 |
| (R)-4-(4-acetylpiperazin-1- yl)-2-(3-(4-amino-3-(2-fluoro- 4-phenoxyphenyl)-1H- pyrazolo[3,4-d]pyrimidin-1- yl)piperidine-1-carbonyl)-4- methylpent-2-enenitrile | 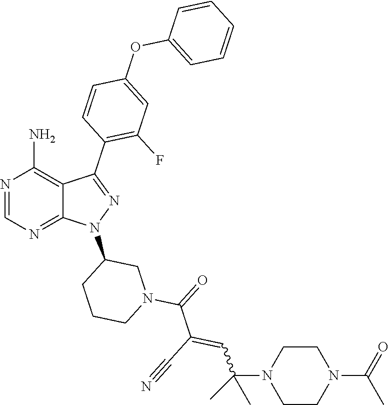 [SMILES: CC(=O)N1CCN(C(C)(C)C=C(C#N)C(=O)N2CCC[C@@H](n3nc(-c4ccc(Oc5ccccc5)cc4F)c4c(N)ncnc43)C2)CC1] | 32 |
| (R)-methyl-4-(5-(3-(4-amino- 3-(2-fluoro-4- phenoxyphenyl)-1H- pyrazolo[3,4-d]pyrimidin-1- yl)piperidin-1-yl)-4-cyano-2- methyl-5-oxopent-3-en-2- yl)piperazine-1-carboxylate | 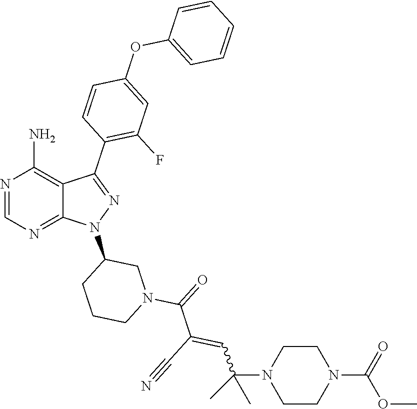 [SMILES: COC(=O)N1CCN(C(C)(C)C=C(C#N)C(=O)N2CCC[C@@H](n3nc(-c4ccc(Oc5ccccc5)cc4F)c4c(N)ncnc43)C2)CC1] | 33 |

In some embodiments, the BTK inhibitor is a compound of Formula (I) or a pharmaceutically acceptable salt thereof, wherein:

Z² is —N—;

—Z-EWG- is

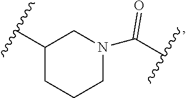 [SMILES: *C(=O)N1CCCC(*)C1]

wherein:

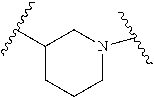 [SMILES: *C1CCCN(*)C1]

is substituted with 0, 1, or 2 substituents independently chosen from alkyl, hydroxy, and halo; and

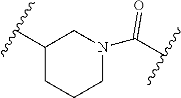 [SMILES: *C(=O)N1CCCC(*)C1]

the carbonyl group in is attached to —C(CN)═CHR^{c}; and

R^{c} is —C(CH₃)₂-(4-R⁸-piperazin-1-yl), wherein R⁸ is oxetan-3-yl.

In some embodiments of the present disclosure, the BTK inhibitor is chosen from (E) isomer, (Z) isomer, and a mixture of (E) and (Z) isomers of 2-[(3R)-3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (Compound (IB)); and/or a pharmaceutically acceptable salt of any of the foregoing compounds.

Compound (IB) is also known as PRN1008 or rilzabrutinib, and has the following chemical structure:

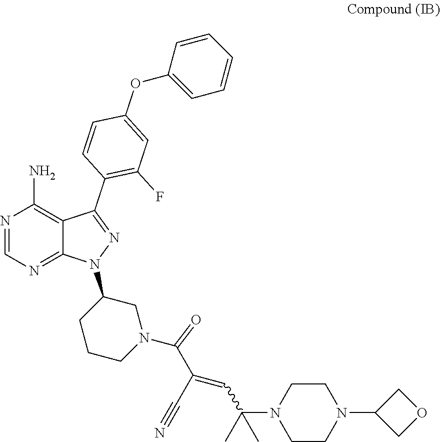 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

The [symbol] line at the alkene carbon in Compound (IB) denotes that Compound (IB) or a pharmaceutically acceptable salt thereof can be (E) isomer, (Z) isomer, or a mixture of (E) and (Z) isomers.

Compound (IB) is disclosed in Example 31 of the PCT Application No. PCT/US2013/058614, filed on Sep. 6, 2013 and published as WO2014/039899. The disclosed synthesis provides Compound (IB) requiring purification by column chromatography and affording a foam upon removal of solvent, which can be crushed to obtain a powder.

Compound (IA), Compound (IB), and pharmaceutically acceptable salts of either are potent Bruton's Tyrosine Kinase (BTK) inhibitors. Compound (IB) is an oral inhibitor of the BTK pathway. It is a reversible covalent inhibitor that is designed to rapidly clear from the circulation, and baseline BTK activity (as measured by occupancy) is recovered within a few days. Compound (IB) is currently in clinical trials for the treatment of both pemphigus vulgaris (PV) and immune thrombocytopenia (ITP).

BTKi provides an alternative immunomodulatory approach for the treatment of COVID-19 patients. Immunomodulation with BTK inhibitors, e.g., compounds of Formula (I), e.g., Compound (IA) or Compound (IB), may be beneficial for the treatment of ARDS and inflammation in COVID-19 patients. BTKi may provide an anti-inflammatory approach to targeting underlying tissue inflammation and detrimental neutrophil and macrophages accumulation in the lung. BTKi may also have anti-thrombotic effects through the inhibition of proinflammatory platelet mechanisms, while sparing normal hemostatic platelet function.

In some embodiments of the present disclosure, at least about 80% w/w, at least about 85% w/w, at least about 90% w/w, at least about 95% w/w, at least about 96% w/w, at least about 97% w/w, or at least about 99% w/w of Compound (IA) or Compound (IB) or a pharmaceutically acceptable salt of either is the (E) isomer. The ratio of the (E) to (Z) isomer can be calculated by methods well known in the art. A non-limiting example of one such method is HPLC total area normalization method.

In some embodiments, the present disclosure provides methods of using at least one compound chosen from compounds of Formula (I) and pharmaceutically acceptable salts thereof as a replacement for corticosteroid therapy for treating a disease chosen from acute respiratory distress syndrome (ARDS), sepsis, sepsis induced acute lung injury, diffuse alveolar damage (DAD), macrophage activation syndrome (MAS), secondary hemophagocytic lymphohistiocytosis (sHIH), cytokine release syndrome (CRS), and systemic inflammatory response syndrome (SIRS).

In some embodiments, the present disclosure provides methods of using at least one compound chosen from compounds of Formula (I) and pharmaceutically acceptable salts thereof as a replacement therapy for treating a disease chosen from acute respiratory distress syndrome (ARDS), sepsis, sepsis induced acute lung injury, diffuse alveolar damage (DAD), macrophage activation syndrome (MAS), secondary hemophagocytic lymphohistiocytosis (sHIH), cytokine release syndrome (CRS), and systemic inflammatory response syndrome (SIRS). In some embodiments, a corticosteroid is used as a first- or second-line therapy for treating the disease. In some embodiments, the at least one compound is used in place of a corticosteroid. In some embodiments, the at least one compound is used in combination with a corticosteroid. In some embodiments, a corticosteroid is used as a first or second line maintenance therapy for the disease. In some embodiments, the at least one compound is used in place of a corticosteroid. In some embodiments, the at least one compound is used in combination with a corticosteroid.

In some embodiments, the present disclosure provides methods of eliminating or reducing a therapeutic dose of a corticosteroid used in chronic maintenance therapy in the treatment of a disease chosen from acute respiratory distress syndrome (ARDS), sepsis, sepsis induced acute lung injury, diffuse alveolar damage (DAD), macrophage activation syndrome (MAS), secondary hemophagocytic lymphohistiocytosis (sHIH), cytokine release syndrome (CRS), and systemic inflammatory response syndrome (SIRS), in a mammal in need thereof, comprising administering to the mammal a therapeutically effective amount of at least one compound chosen from compounds of Formula (I) and pharmaceutically acceptable salts thereof. In some embodiments, the corticosteroid is used as a first- or second-line treatment. In some embodiments, the at least one compound is used in place of the corticosteroid. In some embodiments, the at least one compound is used in combination with the corticosteroid.

In some embodiments of the present disclosure, at least one compound chosen from compounds of Formula (I) and pharmaceutically acceptable salts thereof is administered in combination with a noncorticosteroidal immunosuppressive and/or anti-inflammatory agent. In some embodiments, the at least one compound is administered in combination with an active pharmaceutical ingredient chosen from interferon alpha, interferon gamma, cyclophosphamide, tacrolimus, mycophenolate mofetil, methotrexate, dapsone, sulfasalazine, azathioprine, an anti-CD20 agent (e.g., rituximab, ofatumumab, obinutuzumab, or veltuzumab, or a biosimilar version of any of the foregoing), an anti-TN alpha agent (e.g., etanercept, infliximab, golimumab, adalimumab, or certolizumab pegol, or a biosimilar version of any of the foregoing), an anti-IL6 agent toward ligand or its receptors (e.g., tocilizumab, sarilumab, olokizumab, elsililumab, or siltuximab, or a biosimilar version of any of the foregoing), an anti-IL17 agent to ligand or its receptors (e.g., secukinumab, ustekinumab, brodalumab, or ixekizumab, or a biosimilar version of any of the foregoing), an anti-IL1 agent to ligand or its receptors (e.g., rilonacept, canakinumab, or anakinra, or a biosimilar version of any of the foregoing), an anti-IL2 agent to ligand or its receptors (e.g., basiliximab or daclizumab, or a biosimilar version of either), an anti-CD2 agent (e.g., alefacept or a biosimilar version thereof), an anti-CD3 agent (e.g., muromonab-cd3 or a biosimilar version thereof), an anti-CD80/86 agent (e.g., abatacept or belatacept, or a biosimilar version of either), an anti-sphingosine-1-phosphate receptor agent (e.g., fingolimod or a biosimilar version thereof), an anti-C5 agent (e.g., eculizumab or a biosimilar version thereof), an anti-integrin alpha4 agent (e.g., natalizumab or a biosimilar version thereof), an anti-α₄β₇ agent (e.g., vedolizumab or a biosimilar version thereof), an anti-mTOR agent (e.g., sirolimus or everolimus), an anti-calcineurin agent (e.g., tacrolimus), an anti-BAFF/BlyS agent (e.g., belimumab, VAY736, or blisibimod, or a biosimilar version of any of the foregoing), leflunomide, and teriflunomide.

In some embodiments of the present disclosure, at least one compound chosen from compounds of Formula (I) and pharmaceutically acceptable salts thereof is administered in combination with at least one antiviral agent, e.g., remdesivir. Antiviral agents may include for example, entry inhibitors, uncoating inhibitors, reverse transcriptase inhibitors, ingegrase inhibitors, and protease inhibitors.

### BRIEF DESCRIPTION OF THE DRAWINGS

FIG. 1 illustrates the study design for investigating dose dependent inhibition in an anti-GBM (anti-glomerular basement membrane) mouse glomerulonephritis model with PRN1008 treatment. The mouse anti-GBM glomerulonephritis model involves antibody mediated autoimmunity, and the model is histologically and mechanistically similar to glomerulonephritis in humans. The model also includes kidney deposition of immune complexes (IC), targeting glomerular basement membrane.

FIG. 2 shows dose dependent inhibition of serum blood urea nitrogen (BUN) levels with PRN1008 treatment in a mouse anti-GBM glomerulonephritis model. BUN levels provide a measure of kidney function.

FIG. 3 shows dose dependent inhibition of severe proteinuria with PRN1008 treatment in a mouse anti-GBM glomerulonephritis model.

FIG. 4 shows reduced proteinuria with PRN1008 treatment in a mouse anti-GBM glomerulonephritis model.

FIG. 5 shows dose dependent inhibition of kidney weight gain with PRN1008 treatment in a mouse anti-GBM glomerulonephritis model. Kidney weight gain is a surrogate for kidney inflammation.

FIG. 6 shows that PRN1008 significantly reduced kidney pathology, superior to a steroid comparator (Dex), in a mouse anti-GBM glomerulonephritis model.

In FIGS. 1-6, Dex refers to dexamethasone, a potent synthetic member of the glucocorticoid class of steroid hormones.

FIG. 7 shows a BioMAP Diversity PLUS Panel, which is used in the interpretation of BioMAP biomarker activities relevant to biological pathways and in vivo correlations and predictions.

FIG. 8 depicts a BioMAP Profile of PRN1008.

FIG. 9A shows that the migratory activity of neutrophils was severely decreased in animals treated with Compound (IA) compared to vehicle control in terms of arrested cells that crawled.

FIG. 9B shows representative micrographs of neutrophil (eGFP, green) recruitment to the necrotic zone (propidium iodide, red) 4 h after heat injury as obtained using spinning disc time-lapse microscopy.

### DEFINITIONS

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

As used herein, a “small molecule” refers to an organic compound, wherein the atoms of the compound comprise carbon, hydrogen, oxygen, nitrogen, phosphorus, and sulfur, having a molecular weight of less than 500 g/mol.

As used herein, “Compound (IA)” refers to the (E) isomer, (Z) isomer, or a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile, which has the following structure:

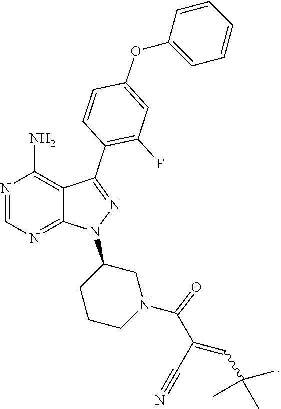 [SMILES: CC(C)(C)C=C(C#N)C(=O)N1CCC[C@@H](n2nc(-c3ccc(Oc4ccccc4)cc3F)c3c(N)ncnc32)C1]

As used herein, “Compound (IB)” refers to the (E) isomer, (Z) isomer, or a mixture of (E) and (Z) isomers of 2-[(3R)-3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile, which has the following structure:

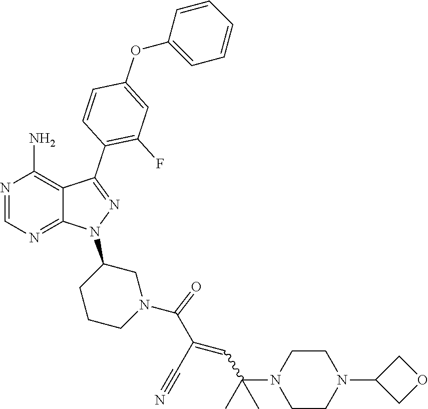 [SMILES: CC(C)(C=C(C#N)C(=O)N1CCC[C@@H](n2nc(-c3ccc(Oc4ccccc4)cc3F)c3c(N)ncnc32)C1)N1CCN(C2COC2)CC1]

or a pharmaceutically acceptable salt thereof.

The [symbol] line at the alkene carbon in Compound (IA) or Compound (IB) denotes that Compound (IA) or Compound (IB) or a pharmaceutically acceptable salt of either can be (E) isomer, (Z) isomer, or a mixture of (E) and (Z) isomers.

All polymorphic forms and hydrates of Compound (IA) and Compound (IB) are within the scope of this disclosure and claims appended hereto.

It will be understood by a person of ordinary skill in the art that when a compound is denoted as the (R) isomer, the compound may contain the corresponding (S) stereoisomer as an impurity, i.e., the (S) stereoisomer in less than about 1% by wt and vice versa.

As used herein, “substantially pure” in connection with a geometric or isomeric form refers to a compound, such as Compound (IA) or Compound (IB), wherein more than 70% by weight of the compound is present as the given isomeric form. For example, the phrase “Compound (IA) is a substantially pure (E) isomer” refers to Compound (IA) having at least 70% by weight or moles of the (E) isomeric form, and the phrase “Compound (IA) is a substantially pure (Z) isomer” refers to Compound (IA) having at least 70% by weight or moles the (Z) isomeric form. The above equally applies to Compound (TB). In some embodiments, at least 80% by weight or moles of Compound (IA) or Compound (IB) is the (E) form or at least 80% by weight or moles of Compound (IA) or Compound (IB) is the (Z) form. In some embodiments, at least 85% by weight or moles of Compound (IA) or Compound (IB) is in the (E) form or at least 85% by weight or moles of Compound (IA) or Compound (IB) is in the (Z) form. In some embodiments, at least 90% by weight or moles of Compound (IA) or Compound (IB) is in the (E) form or at least 90% by weight or moles of Compound (IA) or Compound (IB) is in the (Z) form. In some embodiments, at least 95% by weight or moles of Compound (IA) or Compound (IB) is in the (E) form or at least 95% by weight or moles of Compound (IA) or Compound (IB) is in the (Z) form. In some embodiments, at least 97% by weight or moles, or at least 98% by weight or moles, of Compound (IA) or Compound (IB) is in the (E) form or at least 97% by weight or moles, or at least 98% by weight or moles, of Compound (IA) or Compound (IB) is in the (Z) form. In some embodiments, at least 99% by weight or moles of Compound (IA) or Compound (IB) is in the (E) form or at least 99% by weight or moles of Compound (IA) or Compound (IB) is in the (Z) form. The relative amounts of (E) and (Z) isomers in a solid mixture can be determined according to standard methods and techniques known in the art.

“Acute,” as used herein, means a disease with a rapid onset and/or a short course.

As used herein, a “pharmaceutically acceptable salt” of a compound means a salt that is pharmaceutically acceptable and that possesses the desired pharmacological activity of the parent compound. Such salts include, but are not limited to: acid addition salts, formed with inorganic acids such as hydrochloric acid, hydrobromic acid, sulfuric acid, nitric acid, phosphoric acid, and the like; or formed with organic acids such as formic acid, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or

salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. It is understood that the pharmaceutically acceptable salts are non-toxic.

Additional information on suitable pharmaceutically acceptable salts can be found in Remington's Pharmaceutical Sciences, 17th ed., Mack Publishing Company, Easton, Pa., 1985, portions of which relate to suitable pharmaceutically acceptable salts are incorporated herein by reference. See also Berge at al., Pharmaceutical Salts, Journal of Pharmaceutical Sciences, 1, Volume 66, Number 1, January 1997.

Treatment decisions often follow formal or informal algorithmic guidelines. Treatment options can often be ranked or prioritized into lines of therapy: first-line therapy, second-line therapy, third-line therapy, and so on. First-line therapy is the first therapy that will be tried. Its priority over other options is usually either: (1) formally recommended on the basis of clinical trial evidence for its best-available combination of efficacy, safety, and tolerability; or (2) chosen based on the clinical experience of the physician. If a first-line therapy either fails to resolve the issue or produces intolerable side effects, additional (second line) therapies may be substituted or added to the treatment regimen, followed by third-line therapies, and so on. Accordingly, “first-line” therapy as used herein means a therapy usually given when someone is diagnosed with a particular disease or condition. A first-line therapy could be categorized as standard of care.

“Maintenance therapy,” as used herein, means a therapy, therapeutic regimen, or course of therapy which is administered subsequent to an initial course of therapy administered to a patient with a disease. Maintenance therapy can be used to halt, slow down, or even reverse the progression of the disease, to maintain the improvement in health achieved by the initial treatment and/or enhance the gains achieved by the initial therapy.

A “pharmaceutically acceptable carrier or excipient” means a carrier or an excipient that is useful in preparing a pharmaceutical composition that is generally safe, non-toxic, and neither biologically nor otherwise undesirable, and includes a carrier or an excipient that is acceptable for veterinary use as well as human pharmaceutical use. As used herein, “a pharmaceutically acceptable carrier or excipient” means one or more pharmaceutically acceptable carriers or excipients.

As used herein, “treating,” “treat,” or “treatment” of a disease includes:

(1) inhibiting the disease, i.e., arresting or reducing the development of the disease or its clinical symptoms; or

(2) relieving the disease, i.e., causing regression of the disease or its clinical symptoms.

As used herein, a “therapeutically effective amount” means the amount of a compound of the present disclosure that, when administered to a mammal, e.g., a human, for treating a disease, is sufficient to effect such treatment for the disease. The “therapeutically effective amount” will vary depending on the compound, the disease and its severity and the age, weight, etc., of the mammal to be treated.

As used herein, “QD” means once a day.

As used herein, “BID” means twice a day.

“Mammal,” as used herein, means animals such as dogs, cats, and humans.

Acute respiratory distress syndrome (ARDS) is a condition characterized by shortness of breath, rapid breathing, and/or bluish skin coloration. It is a type of respiratory failure that includes inflammation in the lungs. ARDS impairs the ability of the lungs to exchange oxygen and carbon dioxide. Causes of ARDS may include sepsis, pancreatitis, trauma, pneumonia, and aspiration. In some embodiments of the present disclosure, ARDS is caused by or associated with coronavirus disease 2019 (COVID-19). ARDS caused by COVID-19 is a leading cause of mortality in patients infected by the COVID-19 virus (Mehta 2020; Ryan 2020).

Secondary hemophagocytic lymphohistiocytosis (sHLH) is a hyperinflammatory syndrome that is characterized by fulminant (i.e., severe and sudden in onset) and fatal hypercytokinemia (an immune reaction having a positive feedback loop between cytokines and immune cells, with highly elevated levels of various cytokines) with multiorgan failure (Mehta 2020). sHLH is an acquired form of hemophagocytic lymphohistiocytosis (HLH) that is triggered by an infection, malignancy, autoimmune disease, or other immune challenge. The symptoms of a critical care patient with sHLH are fever, organ dysfunction, lymphadenopathy, and potentially hepato- and/or splenomegaly. In some embodiments of the present disclosure, sHLH is caused by or is associated with COVID-19.

Sepsis (also known as septicemia and blood poisoning) is an inflammatory immune response triggered by an infection. It is a life-threatening condition that is present when the body causes injury to its own tissues and organs while responding to an infection. The infection may be caused by bacteria (most common), fungus, virus, and protozoans. Symptoms of sepsis may include fever, increased heart rate, low blood pressure, increased breathing rate, and confusion. In some embodiments of the present disclosure, the sepsis is caused by or is associated with COVID-19.

Systemic inflammatory response syndrome (SIRS), also known as acute inflammatory syndrome, is an inflammatory condition affecting the whole body. SIRS is the body's response to an infectious or noninfectious assault. SIRS has both pro- and anti-inflammatory components. SIRS is related to systemic inflammation, organ dysfunction, and organ failure, and is a subset of cytokine storm in which there is an abnormal regulation of various cytokines. It is also closely related to sepsis, in which patients satisfy criteria for SIRS and have a suspected or proven infection. Complications of SIRS may include acute kidney injury, shock, and multiple organ dysfunction syndrome. Causes of SIRS may include microbial infections, malaria, trauma, burns, pancreatitis, ischemia, hemorrhage, complications of surgery, adrenal insufficiency, pulmonary embolism, aortic aneurysm, cardiac tamponade, anaphylaxis, and drug overdose. In some embodiments of the present disclosure, SIRS is caused by or is associated with COVID-19.

Cytokine release syndrome (CRS) or cytokine storm syndrome (CSS) is a form of SIRS and can be triggered by a variety of factors such as infections and certain drugs. When it occurs as a result of drug administration, CRS is also known as infusion-related reaction (IRR) or infusion reaction. The syndrome occurs when large numbers of white blood cells are activated and release inflammatory cytokines, which in turn activate more white blood cells. CRS can be an adverse effect of some monoclonal antibody drugs, as well as adoptive T-cell therapies. Symptoms of CRS may include fever, fatigue, loss of appetite, muscle and joint pain, nausea, vomiting, diarrhea, rashes, fast breathing, rapid heartbeat, low blood pressure, seizures, headache, confusion, delirium, hallucinations, tremor, and loss of coordination. In some embodiments of the present disclosure, CRS is caused by or is associated with COVID-19.

Sepsis-induced acute lung injury (ALI) is characterized by edema, inflammatory cell infiltration, and impaired gas exchange. The patient's condition can be aggravated by hypoxia and may lead to multiple organ failure. Approximately 40% of sepsis patients develop ALI. In some embodiments of the present disclosure, sepsis-induced ALI is caused by or is associated with COVID-19.

Diffuse alveolar damage (DAD) is a response to injury in the lung tissue. It consists of intra-alveolar exudate (often described as hyaline membrane) along with hyperplasia of type II pneumocytes which may be cytologically pleomorphic (King 2007). DAD has been observed in autopsy among those dying with AIDS/HIV-1 infection, where possible etiologies may include viral or opportunistic infections (e.g., P. jirovecii), adult respiratory distress syndrome, and oxygen toxicity. Clinically, it is characterized by respiratory distress and diffuse pulmonary infiltrates (Kattan et al. 2012). DAD has been considered the gold standard pathologic finding for ARDS on postmortem examination and lung biopsy (Maley et al. 2020). In some embodiments of the present disclosure, DAD is caused by or is associated with COVID-19.

Macrophage activation syndrome (MAS) is a form of hemophagocytic lymphohistiocytosis (HLH) associated with rheumatologic conditions. It is characterized by hemophagocytosis and overproduction of cytokines, which result from the activation and uncontrolled proliferation of T lymphocytes and macrophages (Manappallil 2016). In some embodiments of the present disclosure, MAS is caused by or is associated with COVID-19. Severe COVID-19 associated pneumonia patients may exhibit features of systemic hyper-inflammation designated under the umbrella term of macrophage activation syndrome (MAS) or cytokine storm, also known as secondary hemophagocytic lymphohistiocytosis (sHLH). This is distinct from HLH associated with immunodeficiency states termed primary HLH—with radically different therapy strategies in both situations. COVID-19 infection with MAS typically occurs in subjects with adult respiratory distress syndrome (ARDS), and historically, non-survival in ARDS was linked to sustained IL-6 and IL-1 elevation (McGonagle et al. 2020).

As used herein, “acyl” refers to a —COR radical, wherein R is chosen from alkyl, haloalkyl, cycloalkyl, cycloalkylalkyl, aryl, aralkyl, heteroaryl, heteroaralkyl, heterocyclyl, and heterocyclylalkyl, and further wherein the aryl, heteroaryl, or heterocyclyl ring either alone or part of another group, e.g., aralkyl, is optionally substituted with one, two, or three substituents independently chosen from alkyl, alkoxy, halo, haloalkoxy, hydroxyl, carboxy, or alkoxycarbonyl, such as, e.g., acetyl, propionyl, benzoyl, or pyridinylcarbonyl. When R is alkyl, the radical is also referred to herein as alkylcarbonyl.

As used herein, “alkyl” refers to a linear saturated monovalent hydrocarbon radical of one to six carbon atoms or a branched saturated monovalent hydrocarbon radical of three to six carbon atoms, such as, e.g., methyl, ethyl, propyl, 2-propyl, butyl (including all isomeric forms), or pentyl (including all isomeric forms).

As used herein, “alkylene” refers to a linear saturated divalent hydrocarbon radical of one to six carbon atoms or a branched saturated divalent hydrocarbon radical of three to six carbon atoms, such as, e.g., methylene, ethylene, propylene, 1-methylpropylene, 2-methylpropylene, butylene, or pentylene.

As used herein, “alkylthio” refers to a —SR radical, wherein R is alkyl, such as, e.g., methylthio or ethylthio.

As used herein, “alkylsulfonyl” refers to a —SO₂R radical, wherein R is alkyl, such as, e.g., methylsulfonyl or ethylsulfonyl.

As used herein, “alkoxy” refers to a —OR radical, wherein R is alkyl, such as, e.g., methoxy, ethoxy, propoxy, or 2-propoxy, n-, iso-, or tert-butoxy.

As used herein, “alkoxyalkyl” refers to a linear monovalent hydrocarbon radical of one to six carbon atoms or a branched monovalent hydrocarbon radical of three to six carbons substituted with at least one alkoxy group, e.g., one or two alkoxy groups, such as, e.g., 2-methoxyethyl, 1-, 2-, or 3-methoxypropyl, or 2-ethoxyethyl.

As used herein, “alkoxycarbonyl” refers to a —C(O)OR radical, wherein R is alkyl, such as, e.g., methoxycarbonyl or ethoxycarbonyl.

As used herein, “aralkyl” refers to a -(alkylene)-R radical, wherein R is aryl.

As used herein, “aryl” refers to a monovalent monocyclic or bicyclic aromatic hydrocarbon radical of 6 to 10 ring atoms, such as, e.g., phenyl or naphthyl.

As used herein, “carboxy” refers to —COOH.

As used herein, “cycloalkyl” refers to a cyclic saturated monovalent hydrocarbon radical of three to ten carbon atoms wherein one or two carbon atoms may be replaced by an oxo group, such as, e.g., cyclopropyl, cyclobutyl, cyclopentyl, or cyclohexyl.

As used herein, “cycloalkylalkyl” refers to a -(alkylene)-R radical, wherein R is cycloalkyl, such as, e.g., cyclopropylmethyl, cyclobutylmethyl, cyclopentylethyl, or cyclohexylmethyl.

As used herein, “cycloalkylene” refers to a cyclic saturated divalent hydrocarbon radical of three to ten carbon atoms wherein one or two carbon atoms may be replaced by an oxo group, such as, e.g., cyclopropylene, cyclobutylene, cyclopentylene, or cyclohexylene.

As used herein, “halo” refers to fluoro, chloro, bromo, or iodo.

As used herein, “haloalkyl” refers to an alkyl radical as defined above, which is substituted with one or more halogen atoms, e.g., one to five halogen atoms, such as fluorine or chlorine, including those substituted with different halogens, such as, e.g., —CH₂Cl, —CF₃, —CHF₂, —CH₂CF₃, —CF₂CF₃, or —CF(CH₃)₂.

As used herein, “haloalkoxy” refers to a —OR radical, wherein R is a haloalkyl.

As used herein, “heteroaralkyl” refers to a -(alkylene)-R radical, wherein R is heteroaryl.

As used herein, “heteroaryl” refers to a monovalent monocyclic or bicyclic aromatic radical of 5 to 10 ring atoms where one or more, e.g., two or three, ring atoms are heteroatoms independently chosen from N, O, and S, the remaining ring atoms being carbon. Non-limiting examples include pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, and tetrazolyl.

As used herein, “heterocyclyl” refers to a saturated or unsaturated monovalent monocyclic group of 4 to 8 ring atoms in which one or two ring atoms are heteroatoms independently chosen from N, O, and S(O)ₙ, wherein n is an integer from 0 to 2, the remaining ring atoms being C. The heterocyclyl ring is optionally fused to one aryl or heteroaryl ring provided the aryl and heteroaryl rings are monocyclic. A heterocyclyl ring fused to a monocyclic aryl or heteroaryl ring is also referred to herein as a “bicyclic heterocyclyl” ring. Additionally, one or two ring carbon atoms in the heterocyclyl ring can optionally be replaced by a —CO— group. Non-limiting examples of heterocyclyls include pyrrolidino, piperidino, homopiperidino, 2-oxopyrrolidinyl, 2-oxopiperidinyl, morpholino, piperazino, tetrahydropyranyl, and thiomorpholino groups. When the heterocyclyl ring is unsaturated, it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl.

As used herein, “heterocyclylalkyl” refers to a -(alkylene)-R radical, wherein R is heterocyclyl, such as, e.g., tetraydrofuranylmethyl, piperazinylmethyl, or morpholinylethyl.

As used herein, “hydroxyalkyl” refers to a linear monovalent hydrocarbon radical of one to six carbon atoms or a branched monovalent hydrocarbon radical of three to six carbons substituted with one or two hydroxy groups, provided that if two hydroxy groups are present they are not both on the same carbon atom. Non-limiting examples include hydroxymethyl, 2-hydroxyethyl, 2-hydroxypropyl, 3-hydroxypropyl, 1-(hydroxymethyl)-2-methylpropyl, 2-hydroxybutyl, 3-hydroxybutyl, 4-hydroxybutyl, 2,3-dihydroxypropyl, 1-(hydroxymethyl)-2-hydroxyethyl, 2,3-dihydroxybutyl, 3,4-dihydroxybutyl and 2-(hydroxymethyl)-3-hydroxypropyl, preferably 2-hydroxyethyl, 2,3-dihydroxypropyl, and 1-(hydroxymethyl)-2-hydroxyethyl.

As used herein, “oxo” or “carbonyl” refers to C═(O) group.

As used herein, “substituted alkyl” refers to an alkyl group substituted with one, two, or three substituents independently chosen from hydroxyl, alkoxy, carboxy, cyano, carboxy, alkoxycarbonyl, alkylthio, alkylsulfonyl, halo, —CONRR, —NRR, and heterocyclyl (e.g., heterocycloamino), wherein:
each R is independently chosen from hydrogen, alkyl, cycloalkyl, hydroxyalkyl, and alkoxyalkyl;
each R′ is independently chosen from hydrogen, alkyl, and cycloalkyl;
the heterocyclyl group is optionally substituted with one or two groups independently chosen from acyl, alkyl, alkylthio, alkylsulfonyl, alkoxy, alkoxyalkyl, alkoxycarbonyl, halo, haloalkyl, heterocyclyl, hydroxyl, and —CONR*R″;
R* is chosen from hydrogen, alkyl, cycloalkyl, and hydroxyalkyl; and
R″ is chosen from hydrogen, alkyl, and cycloalkyl.

### Formulations and Administration:

In general, the compounds of this disclosure will be administered in a therapeutically effective amount by any of the accepted modes of administration (e.g., oral administration) for agents that serve similar utilities. Therapeutically effective amounts of compounds of this disclosure may range from about 0.01 to about 500 mg per kg patient body weight per day, which can be administered in single or multiple doses. A suitable dosage level may be from about 0.1 to about 250 mg/kg per day, such as about 0.5 to about 100 mg/kg per day.

A suitable dosage level may also be about 0.01 to about 250 mg/kg per day, such as about 0.05 to about 100 mg/kg per day, and further such as about 0.1 to about 50 mg/kg per day. Within this range, the dosage can be about 0.05 to about 0.5, such as about 0.5 to about 5, and further such as about 5 to about 50 mg/kg per day. For oral administration, the compositions can be provided in the form of tablets containing about 1 to about 1000 milligrams of the active ingredient, particularly about 1, 5, 10, 15, 20, 25, 50, 75, 100, 150, 200, 250, 300, 400, 500, 600, 750, 800, 900, and 1000 milligrams of the active ingredient. The actual amount to be administered of the compound of this disclosure, i.e., the active ingredient, will depend upon numerous factors such as the severity of the disease to be treated, the age and relative health of the patient, the potency of the compound being utilized, the route and form of administration, and other factors.

In general, compounds of this disclosure will be administered as pharmaceutical compositions by any one of the following routes: oral, systemic (e.g., transdermal, intranasal or by suppository), or parenteral (e.g., intramuscular, intravenous, or subcutaneous) administration. The preferred manner of administration is oral using a convenient daily dosage regimen, which can be adjusted according to the degree of affliction. Compositions can take the form of tablets, pills, capsules, semisolids, powders, sustained release formulations, solutions, suspensions, elixirs, aerosols, or any other appropriate compositions.

The choice of formulation depends on various factors such as the mode of drug administration (e.g., for oral administration, formulations in the form of tablets, pills or capsules, including enteric coated or delayed release tablets, pills or capsules are preferred) and the bioavailability of the drug substance. Recently, pharmaceutical formulations have been developed especially for drugs that show poor bioavailability based upon the principle that bioavailability can be increased by increasing the surface area, i.e., decreasing particle size. For example, U.S. Pat. No. 4,107,288 describes a pharmaceutical formulation having particles in the size range from 10 to 1,000 nm in which the active material is supported on a cross-linked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability. The portions of both patents related to pharmaceutical formulations are incorporated by reference herein.

In general, the compositions comprise a compound of this disclosure in combination with a pharmaceutically acceptable excipient. Pharmaceutically acceptable excipients are non-toxic, aid administration, and do not adversely affect the therapeutic benefit of the compound of this disclosure. Such excipients may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be chosen from glycerol, propylene glycol, water, ethanol, and various oils, including those of petroleum, animal, vegetable, or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this disclosure in aerosol form. Inert gases suitable for this purpose are nitrogen, carbon dioxide, etc.

Other suitable pharmaceutical excipients and their formulations are described in Remington's Pharmaceutical Sciences, edited by E. W. Martin (Mack Publishing Company, 20^{th} ed., 2000), which is incorporated by reference herein with respect to the portions relating to pharmaceutical excipients and their formulations.

The level of the compound in a formulation can vary within the full range employed by those skilled in the art. Typically, the formulation will contain, on a weight percent (wt. %) basis based on the total formulation, from about 0.01-99.99 wt. % of a compound of this disclosure, with the balance being a suitable pharmaceutical excipients. For example, the compound is present at a level of about 1-80 wt. %. With respect to the numerical range 0.01-99.99, “about” denotes less than 0.01%. With respect to the numerical range 1 to 80, “about” denotes 0.05 with respect to 1 and 10 with respect to 80, thus covering a range from 0.05 to 90 wt. %.

The compounds of this disclosure may be used in combination with one or more other drugs in the treatment of diseases or conditions for which compounds of this disclosure or the other drugs may have utility. Such other drug(s) may be administered, by a route and in an amount commonly used therefore, contemporaneously, such as fixed dose combination, or sequentially with a compound of the present disclosure. When a compound of this disclosure is used contemporaneously with one or more other drugs, a pharmaceutical composition in unit dosage form containing such other drugs and the compound of the present disclosure, i.e., a fixed dose compound, is preferred. However, the combination therapy may also include therapies in which the compound of this disclosure and one or more other drugs are administered on different overlapping schedules or even non-overlapping schedules. It is also contemplated that situations will arise that when used in combination with one or more other active ingredients, the compounds of the present disclosure and the other active ingredients may be used in lower doses than when each is used singly.

All publications and patents mentioned herein are hereby incorporated by reference in their entirety as if each individual publication or patent was specifically and individually indicated to be incorporated by reference unless a certain portion of the disclosure is specifically incorporated.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and permutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

### EXAMPLES

The following examples are intended to be illustrative and are not meant in any way to limit the scope of the disclosure.

### Example 1: Mouse Anti-GBM Glomerulonephritis Model

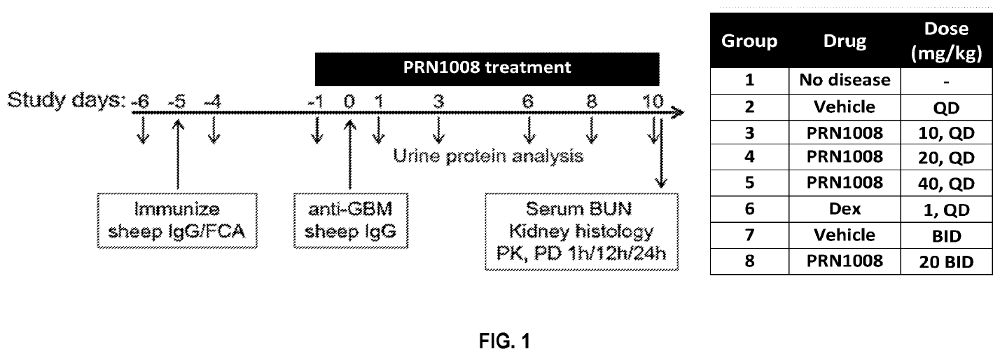

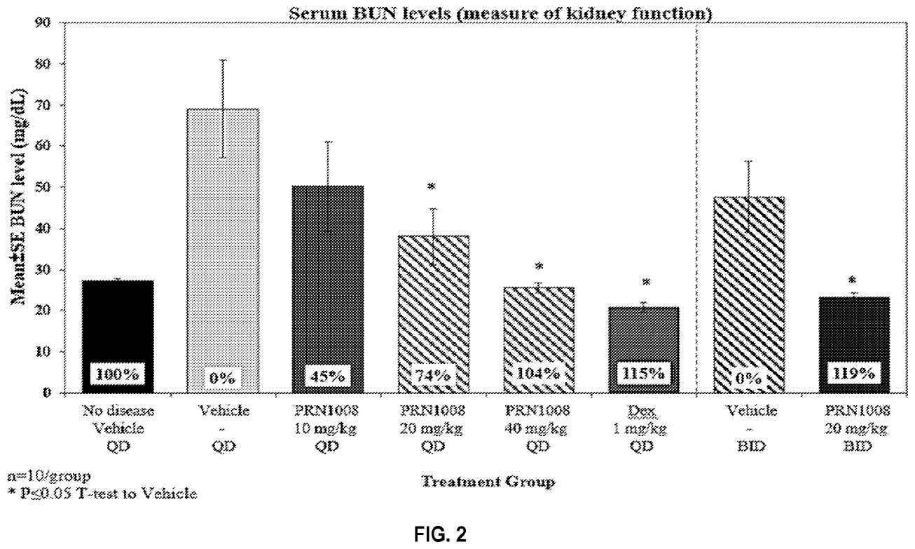

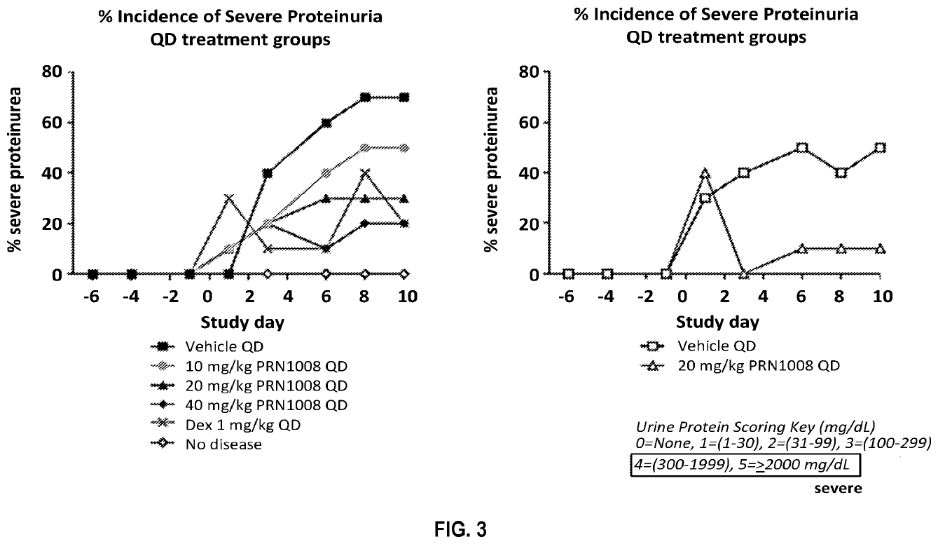

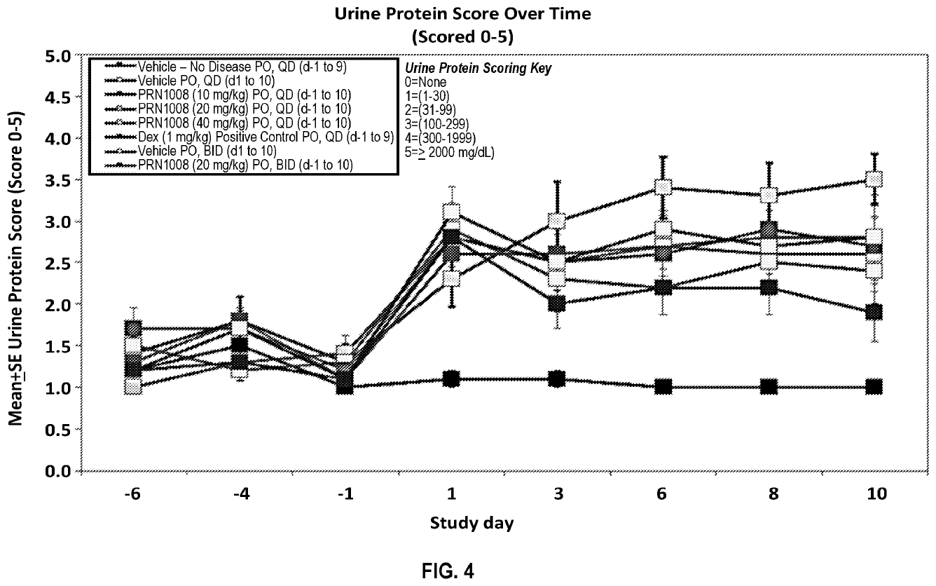

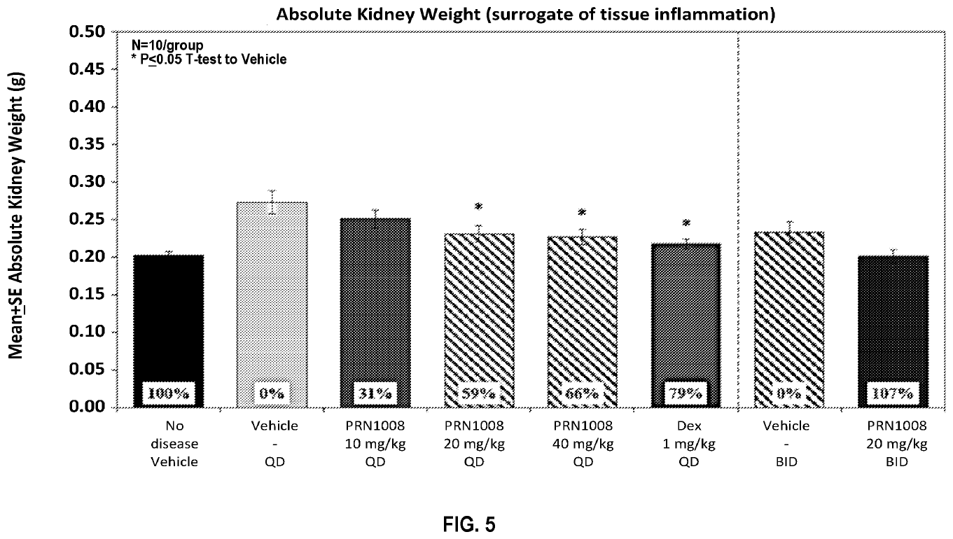

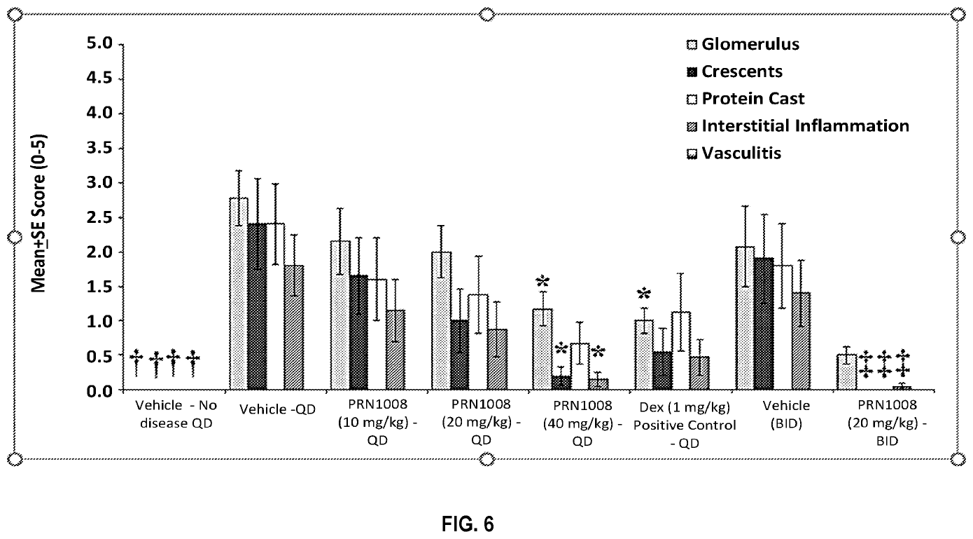

The efficacy of Compound (IB) (also referred to as PRN1008) relative to the corticosteroid dexamethasone (Dex) was tested in a mouse anti-GBM glomerulonephritis model according to the design shown in FIG. 1. Briefly, to induce glomerulonephritis, mice were pre-sensitized with sheep IgG/FCA (Study Day −5). Five days later (Study Day 0), the mice received anti-GBM sheep IgG. Treatment with vehicle, Compound (IB), or Dex with various dosage regimes (Compound (IB): 10 mg/kg, 20 mg/kg, or 40 mg/kg QD or 20 mg/kg BID; Dex: 1 mg/kg QD; Vehicle: QD or BID) began at Study Day −1, one day prior to injection with anti-GBM sheep IgG. Treatment continued until Study Day 10, for eleven days treatment in total. Urine protein analysis was conducted on Study Days −6, −4, −1, 1, 3, 6, 8, and 10. Following Study Day 10, mice serum BUN levels were analyzed as a measure of kidney function, and kidney histology was performed. In the mouse anti-GBM glomerulonephritis model, dose dependent inhibition of serum BUN levels (FIG. 2), severe proteinuria (FIG. 3), and kidney weight gain (a surrogate for kidney inflammation) (FIG. 5) were observed. Additionally, treatment with Compound (IB) led to reduced proteinuria during the study (FIG. 4), and Compound (IB) reduced kidney pathology (FIG. 6), providing favorable results relative to Dex.

### Example 2: BioMAP Diversity PLUS Panel

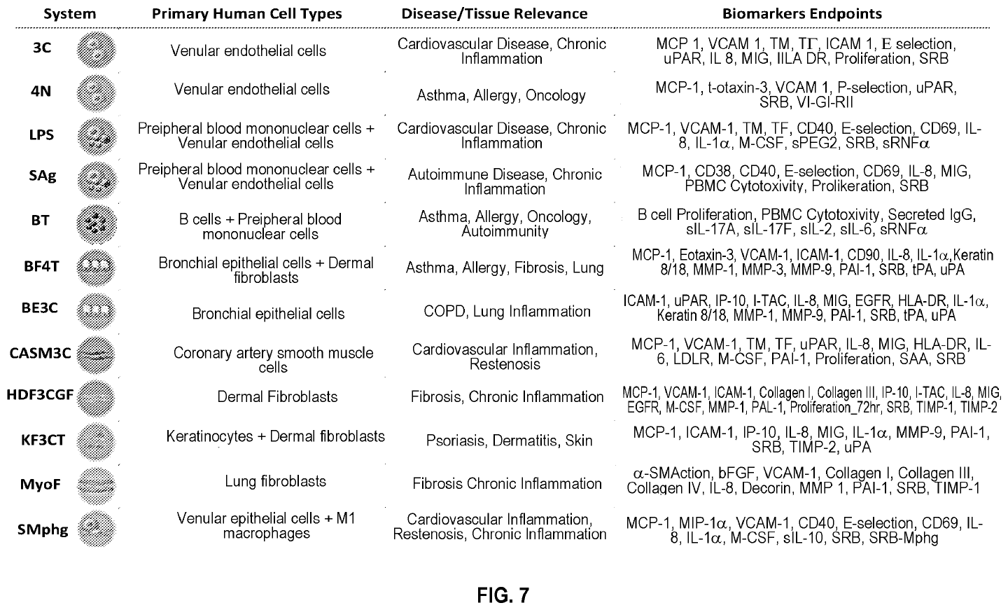

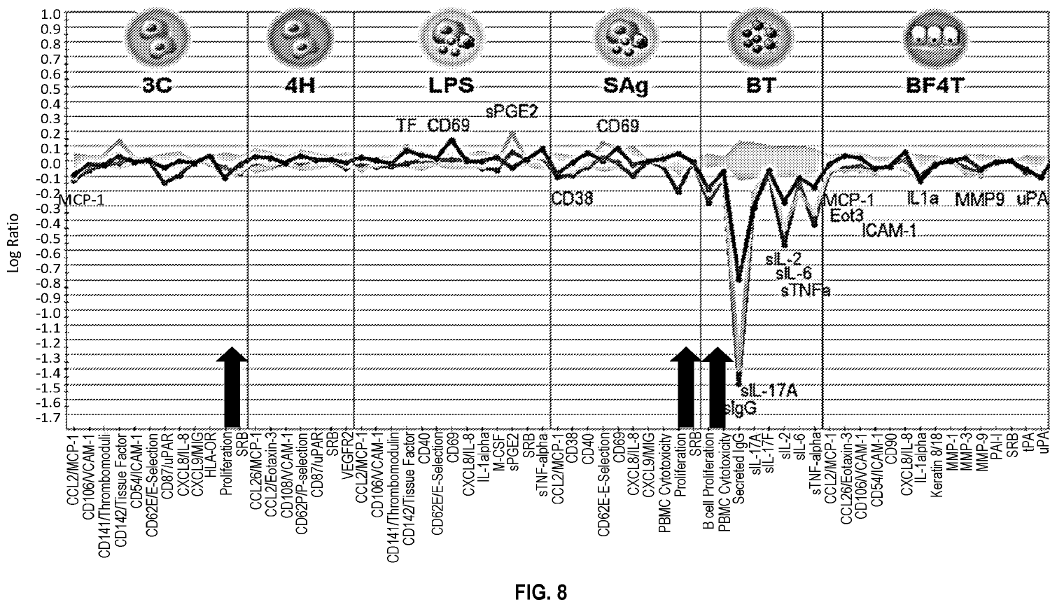

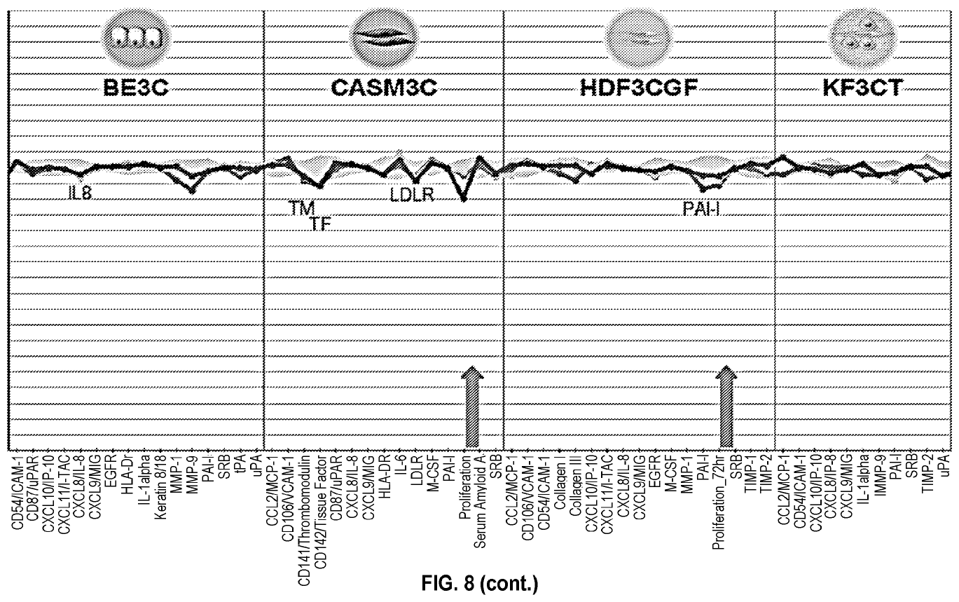

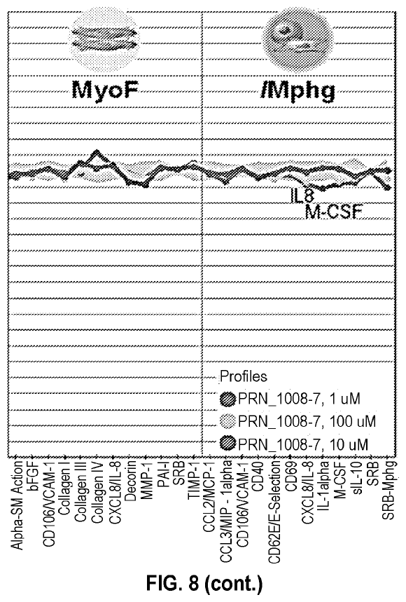

The BioMAP Diversity Plus Panel (FIG. 7) provides broad phenotypic profiles for pharmaceutically active agents. The panel uses 12 individual BioMAP human primary cell-based co-culture systems to predictively model drug effects on multiple tissues and disease states, providing 148 clinically relevant biomarker readouts. Some key activities of PRN1008 illustrated by the BioMAP Profile (FIG. 8) include anti-proliferative activities, inflammation-related activities, immunomodulatory activities, tissue remodeling activities, hemostasis-related activities, and decreased LDLR. The LDLR gene is associated with the low-density lipoprotein receptor, which binds low-density lipoproteins that carry cholesterol in blood. Anti-proliferative activities with respect to endothelial cells, T cells, B cells, coronary artery smooth muscle cells, and fibroblasts are indicated by grey arrows. Decreased MCP-1, sTNFα, eotaxin-3, ICAM-1, IL-la, and IL-8 and increased sPGE2 is associated with PRN1008's inflammation related activities. Decreased CD38, sIgG, sIL-17A, sIL-2, sIL-6, and M-CSF and increased CD69 are associated with PRN1008's immunomodulatory activities. Decreased MMP-9, uPA, and PAI-I is associated with PRN 1008's tissue remodeling activities. Decreased thrombomodulin (TM) and modulated tissue factor (TF) is associated with PRN1008's hemostasis-related activities. Thus, the BioMAP Diversity Plus Panel data for PRN1008 supports its anti-inflammatory and cytokine inhibition mechanisms.

### Example 3: Neutrophil Migration Study

Following post-adhesion strengthening, neutrophils undertake Mac-1 (an integrin) dependent migratory activity on the vascular side of the vessel wall prior to transmigration (Herter and Zarbock 2013). Previous studies have highlighted the importance of this step for successful neutrophil recruitment (Phillipson et al. 2006). To investigate the effects of Btk inhibition on this step of the leukocyte recruitment cascade, intraluminal crawling following fMLP (N-formylmethionine-leucyl-phenylalanine)-mediated arrest in vivo was investigated.

Using intravital microscopy, intravascular crawling of neutrophils was examined as described previously (Phillipson et al. 2006). Briefly, anti-Gr-1 antibody (clone RB6-8C5), labelled with Alexa Fluor 488 (Molecular Probes, Eugene, Oreg., USA), was injected via the cannulated carotid artery prior to the experiment. Following preparation and exteriorization, the cremaster was superfused with fMLP (10 μM) and time-lapse microscopy was performed for 2 h. The number of adherent cells was determined.

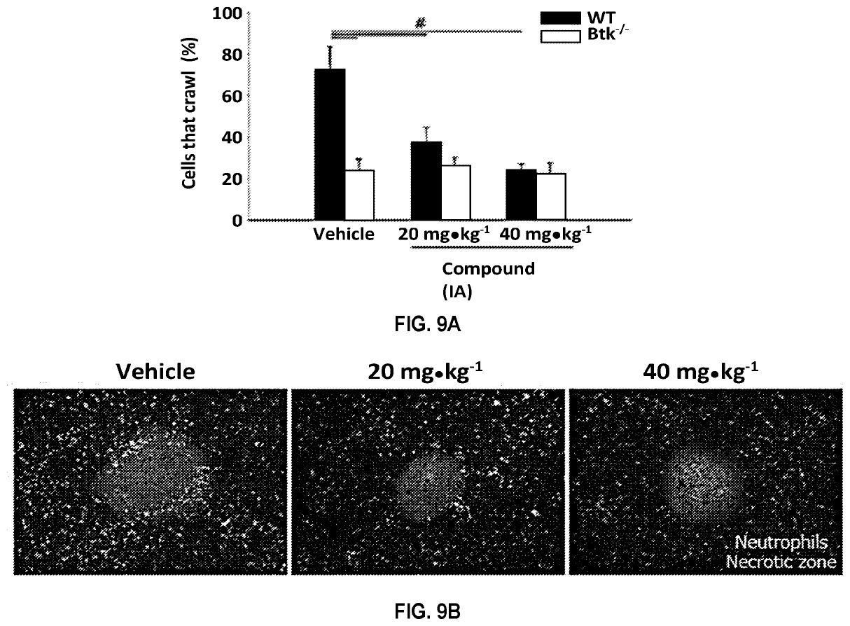

Migratory activity of neutrophils was severely decreased in animals treated with Compound (IA) compared to vehicle control in terms of arrested cells that crawled (FIG. 9A). Compound (IA) was also found to abolish neutrophil recruitment following sterile liver injury (FIG. 9B). FIG. 9B shows representative micrographs of neutrophil (eGFP, green) recruitment to the necrotic zone (propidium iodide, red) 4 h after heat injury as obtained using spinning disc time-lapse microscopy.

### REFERENCES

Busygina K, et al. Oral Bruton tyrosine kinase inhibitors selectively block atherosclerotic plaque-triggered thrombus formation in humans. Blood. 2018. 131(24):2605-2616. https://doi.org/10.1182/blood-2017-09-808808
Cao X. COVID-19: imtrunopathology and its implications for therapy. Nat Rev Immunol. 2020. https://doi.org/10.1038/s41577-020-0308-3
DePorto A P, et al. Btk inhibitor ibrutinib reduces inflammatory myeloid cell responses in the lung during murine pneumococcal pneumonia. Mol Med. 2019. 25(3). https://doi.org/10.1186/s10020-018-0069-7
Florence J M, et al. Inhibiting Bruton's tyrosine kinase rescues mice from lethal influenza-induced acute lung injury. Am J Physiol Lung Cell Mol Physiol. 2018. 315(1):L52-L58. https://doi.org/10.1152/ajplung.00047.2018
Herter J M, et al. PRN473, an inhibitor of Bruton's tyrosine kinase, inhibits neutrophil recruitment via inhibition of macrophage antigen-1 signalling. Br J Pharmacol, 2018, 175(3):429-439. https://doi.org/10.1111/bph.14090
Herter J M, et al. (2013). Integrin regulation during leukocyte recruitment. J Immunol 190: 4451-4457.
Huang X, et al. The Role of Macrophages in the Pathogenesis of ALI/ARDS. Mediators Inflamm. 2018. https://doi.org/10.1155/2018/1264913
Kattan M, et al. “Respiratory Disorders in Pediatric HIV Infection” in Kendig & Chernick's Disorders of the Respiratory Tract in Children (Eighth Edition), 2012
King T, “Respiratory Tract and Pleura” in Elsevier's Integrated Pathology, 2007
Krupa A, et al. Silencing Bruton's tyrosine kinase in alveolar neutrophils protects mice from LPS/immune complex-induced acute lung injury. Am J Physiol Lung Cell Mol Physiol. 307(6): L435-L448, 2014.
Manappallil R, “A Case of Macrophage Activation Syndrome with Acute Respiratory Distress Syndrome” Journal of Clinical and Diagnostic Research. 2016 September, Vol-10(9): OD11-OD12 McGonagle D, et al. “The Role of Cytokines including Interleukin-6 in COVID-19 induced Pneumonia and Macrophage Activation Syndrome-Like Disease.” Autoimmun Rev. 2020 Apr. 3:102537. doi: 10.1016/j.autrev.2020.102537
Mehta P, et al. COVID-19: consider cytokine storm syndromes and immunosuppression. The Lancet. 2020. 395 (10229):1033-1034. https://doi.org/10.1016/S0140-6736(20)30628-0
Murphy S, et al. Care for Critically Ill Patients With COVID-19. JAMA Insights. 2020. https://doi.org/10.1001/jama.2020.3633
Phillipson M et al. (2006). Intraluminal crawling of neutrophils to emigration sites: a molecularly distinct process from adhesion in the recruitment cascade. J Exp Med 203: 2569-2575.
Rip J, et al. The role of Bruton's Tyrosine Kinase in immune cell signaling and systemic autoimmunity. Crit Rev Immunol, 38(1):17-62, 2018. https://doi.org/10.1615/CritRevImmunol.2018025184.
Thachil J, et al. ISTH interim guidance on recognition and management of coagulopathy in COVID-19. J Thromb Hemostasis. 2020. https://doi.org/10.1111/JTH.14810
Zhang D, et al. COVID-19 infection induces readily detectable morphological and inflammation-related phenotypic changes in peripheral blood monocytes, the severity of which correlate with patient outcome. 2020. https://doi.org/10.1101/2020.03.24.20042655
Jason H. Maley, B. Taylor Thompson, “ARDS: Are the current definitions useful?”, in Evidence-Based Practice of Critical Care (Third Edition), 2020.

What is claimed:

1. A method of treating a disease chosen from acute respiratory distress syndrome, sepsis, sepsis induced acute lung injury, diffuse alveolar damage, macrophage activation syndrome, secondary hemophagocytic lymphohistiocytosis, cytokine release syndrome, and systemic inflammatory response syndrome comprising administering to a mammal in need of such treatment a pharmaceutical composition comprising a Bruton's tyrosine kinase (BTK) inhibitor and a pharmaceutically acceptable carrier or excipient, wherein the BTK inhibitor is a small molecule.

2. The method according to claim 1, wherein the BTK inhibitor is a compound of Formula (I) or a pharmaceutically acceptable salt thereof: 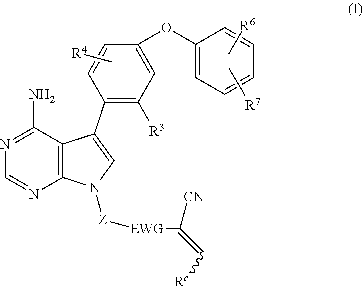
wherein:
Z² is —N— or CR², wherein R² is chosen from hydrogen and alkyl;
R³ and R⁴ are independently chosen from hydrogen, methyl, chloro, fluoro, cyclopropyl, hydroxy, methoxy, cyano, trifluoromethyl, and trifluoromethoxy;
R⁶ and R⁷ are independently chosen from hydrogen, methyl, methoxy, fluoro, chloro, trifluoromethyl, trifluoromethoxy, and cyano;
Z-EWG- is chosen from -alkylene-NR′CO—, -alkylene-NR′SO₂—, 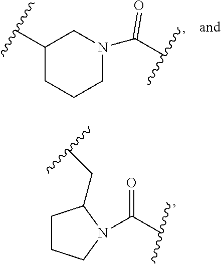 [SMILES: *C(=O)N1CCCC(*)C1 ; *CC1CCCN1C(*)=O]
wherein:
each of 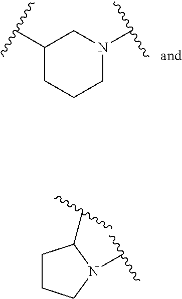 [SMILES: *C1CCCN(*)C1 ; *C1CCCN1*]
and is independently substituted with 0, 1, or 2 substituents independently chosen from alkyl, hydroxy, and halo;
the carbonyl or the sulfonyl group in -alkylene-NR′CO—, -alkylene-NR′SO₂—, 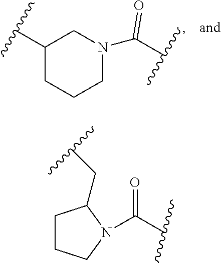 [SMILES: *C(=O)N1CCCC(*)C1 ; *CC1CCCN1C(*)=O]
is attached to —C(CN)═CHR^{c}; and
R′ is independently chosen from hydrogen and alkyl;
R^{c} is chosen from alkyl, haloalkoxy, substituted alkyl, cycloalkyl, cycloalkylene-NR^{d}R^{e}, and cycloalkylene-alkylene-NR^{d}R^{e}; and
R^{d} and R^{e} are independently chosen from hydrogen, alkyl, cycloalkyl, and 3 to 6 membered saturated monocyclic heterocyclyls, wherein:
the heterocyclyls comprise one or two heteroatoms independently chosen from N, O, and S; and
the heterocyclyls are substituted with 0, 1, or 2 substituents independently chosen from hydroxy, alkyl and fluoro.

3. The method according to any one of claims 1-2, wherein Z² is —N—.

4. The method according to any one of claims 1-3, wherein:
Z-EWG- is 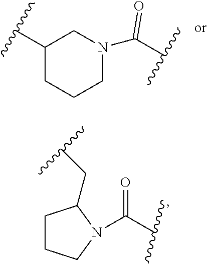 [SMILES: *C(=O)N1CCCC(*)C1 ; *CC1CCCN1C(*)=O]
wherein: 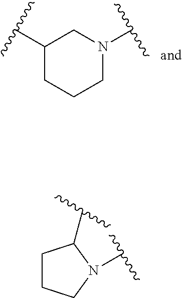 [SMILES: *C1CCCN(*)C1 ; *C1CCCN1*]
and are independently substituted with 0, 1, or 2 substituents independently chosen from alkyl, hydroxy, and halo, and
the carbonyl group in 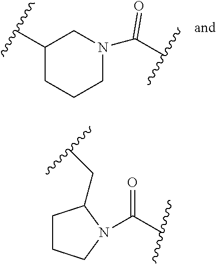 [SMILES: *C(=O)N1CCCC(*)C1 ; *CC1CCCN1C(*)=O]
is attached to —C(CN)═CHR^{c}.

5. The method according to any one of claims 1-4, wherein —Z-EWG- is

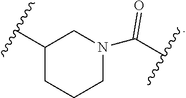 [SMILES: *C(=O)N1CCCC(*)C1]

6. The method according to claim 5, wherein the BTK inhibitor is chosen from (E) isomer, (Z) isomer, and a mixture of (E) and (Z) isomers of:
(R)-2-(3-(4-amino-3-(4-phenoxyphenyl)-1H-pyrazolo[3,4-d]pyrimidin-1-yl)piperidine-1-carbonyl)-3-cyclopropylacrylonitrile;
2-((R)-3-(4-amino-3-(4-(3,4-dichlorophenoxy)-3-methoxyphenyl)-1H-pyrazolo[3,4-d]pyrimidin-1-yl)piperidine-1-carbonyl)-3-cyclopropylacrylonitrile;
(R)-2-(3-(4-amino-3-(4-phenoxyphenyl)-1H-pyrazolo[3,4-d]pyrimidin-1-yl)piperidine-1-carbonyl)-4,4-dimethylpent-2-enenitrile;
(R)-2-(3-(4-amino-3-(4-(3,4-dichlorophenoxy)-3-methoxyphenyl)-1H-pyrazolo-[3,4-d]pyrimidin-1-yl)piperidine-1-carbonyl)-4,4-dimethylpent-2-enenitrile;
(R)-2-(3-(4-amino-3-(4-(2-fluorophenoxy)phenyl)-1H-pyrazolo[3,4-d]pyrimidin-1-yl)piperidine-1-carbonyl)-3-cyclopropylacrylonitrile;
(R)-2-(3-(4-amino-3-(4-(3-fluorophenoxy)phenyl)-1H-pyrazolo[3,4-d]pyrimidin-1-yl)piperidine-1-carbonyl)-3-cyclopropylacrylonitrile;
(R)-2-(3-(4-amino-3-(4-(2,3-difluorophenoxy)phenyl)-1H-pyrazolo[3,4-d]pyrimidin-1-yl)piperidine-1-carbonyl)-3-cyclopropylacrylonitrile;
(R)-2-(3-(4-amino-3-(4-(2,6-difluorophenoxy)phenyl)-1H-pyrazolo[3,4-d]pyrimidin-1-yl)piperidine-1-carbonyl)-3-cyclopropylacrylonitrile;
(R)-2-(3-(4-amino-3-(4-(3,5-difluorophenoxy)phenyl)-1H-pyrazolo[3,4-d]pyrimidin-1-yl)piperidine-1-carbonyl)-3-cyclopropylacrylonitrile;
(R)-2-(3-(4-amino-3-(4-(2,5-difluorophenoxy)phenyl)-1H-pyrazolo[3,4-d]pyrimidin-1-yl)piperidine-1-carbonyl)-3-cyclopropylacrylonitrile;
(R)-2-(3-(4-amino-3-(2-fluoro-4-(phenoxy)phenyl)-1H-pyrazolo[3,4-d]pyrimidin-1-yl)piperidine-1-carbonyl)-3-cyclopropylacrylonitrile;
(R)-2-(3-(4-amino-5-(4-phenoxyphenyl)-7H-pyrrolo[2,3-d]pyrimidin-7-yl)piperidine-1-carbonyl)-3-cyclopropylacrylonitrile; and
(R)-2-(3-(4-amino-3-(4-(2,3-difluorophenoxy)-2-fluorophenyl)-1H-pyrazolo[3,4-d]pyrimidin-1-yl)piperidine-1-carbonyl)-4-methylpent-2-enenitrile;
or a pharmaceutically acceptable salt of any of the foregoing compounds.

7. The method according to any one of claims 1-4, wherein —Z-EWG- is

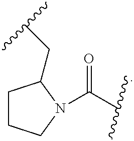 [SMILES: *CC1CCCN1C(*)=O]

8. The method according to claim 7, wherein the BTK inhibitor is chosen from (E) isomer, (Z) isomer, and a mixture of (E) and (Z) isomers of:
2-(2-((4-amino-3-(4-phenoxyphenyl)-1H-pyrazolo[3,4-d]pyrimidin-1-yl)methyl)pyrrolidine-1-carbonyl)-4,4-dimethylpent-2-enenitrile;
(R)-2-(2-((4-amino-3-(4-(3-fluorophenoxy)phenyl)-1H-pyrazolo[3,4-d]pyrimidin-1-yl)methyl)pyrrolidine-1-carbonyl)-3-cyclopropylacrylonitrile;
(R)-2-(2-((4-amino-3-(4-(2,6-difluorophenoxy)phenyl)-1H-pyrazolo[3,4-d]pyrimidin-1-yl)methyl)pyrrolidine-1-carbonyl)-3-cyclopropylacrylonitrile;
(R)-2-(2-((4-amino-3-(2-fluoro-4-phenoxyphenyl)-1H-pyrazolo[3,4-d]pyrimidin-1-yl)methyl)pyrrolidine-1-carbonyl)-3-cyclopropylacrylonitrile;
(R)-2-(2-((4-amino-3-(4-(2,3-difluorophenoxy)phenyl)-1H-pyrazolo[3,4-d]pyrimidin-1-yl)methyl)pyrrolidine-1-carbonyl)-3-cyclopropylacrylonitrile;
(R)-2-(2-((4-amino-3-(4-(3,5-difluorophenoxy)phenyl)-1H-pyrazolo[3,4-d]pyrimidin-1-yl)methyl)pyrrolidine-1-carbonyl)-3-cyclopropylacrylonitrile;
(R)-2-(2-((4-amino-3-(4-(2-fluorophenoxy)phenyl)-1H-pyrazolo[3,4-d]pyrimidin-1-yl)methyl)pyrrolidine-1-carbonyl)-3-cyclopropylacrylonitrile;
(S)-2-{2-[4-amino-5-(4-phenoxy-phenyl)-pyrrolo[2,3-d]pyrimidin-7-ylmethyl]-pyrrolidine-1-carbonyl}-3-cyclopropyl-acrylonitrile;
(S)-2-(2-((4-amino-6-methyl-5-(4-phenoxyphenyl)-7H-pyrrolo[2,3-d]pyrimidin-7-yl)methyl)pyrrolidine-1-carbonyl)-3-cyclopropylacrylonitrile;
(S)-2-(2-((4-amino-6-methyl-5-(4-phenoxyphenyl)-7H-pyrrolo[2,3-d]pyrimidin-7-yl)methyl)pyrrolidine-1-carbonyl)-4-(dimethylamino)-4-methylpent-2-enenitrile;
(S)-2-(2-((4-amino-3-(2-fluoro-4-phenoxyphenyl)-1H-pyrazolo[3,4-d]pyrimidin-1-yl)methyl)pyrrolidine-1-carbonyl)-3-cyclopropylacrylonitrile;
(R)-2-(2-((4-amino-3-(4-(2,3-difluorophenoxy)-2-fluorophenyl)-1H-pyrazolo[3,4-d]pyrimidin-1-yl)methyl)pyrrolidine-1-carbonyl)-3-cyclopropylacrylonitrile;
(S)-2-(2-((4-amino-3-(4-(2,3-difluorophenoxy)-2-fluorophenyl)-1H-pyrazolo[3,4-d]pyrimidin-1-yl)methyl)pyrrolidine-1-carbonyl)-3-cyclopropylacrylonitrile;
2-((S)-2-((4-amino-3-(2-fluoro-4-phenoxyphenyl)-1H-pyrazolo[3,4-d]pyrimidin-1-yl)methyl)pyrrolidine-1-carbonyl)-4-(ethylamino)-4-methylpent-2-enenitrile;
2-((R)-2-((4-amino-3-(2-fluoro-4-phenoxyphenyl)-1H-pyrazolo[3,4-d]pyrimidin-1-yl)methyl)pyrrolidine-1-carbonyl)-4-(ethylamino)-4-methylpent-2-enenitrile;
2-((S)-2-((4-amino-3-(2-fluoro-4-phenoxyphenyl)-1H-pyrazolo[3,4-d]pyrimidin-1-yl)methyl)pyrrolidine-1-carbonyl)-4-(cyclopropylamino)-4-methylpent-2-enenitrile;
2-((S)-2-((4-amino-3-(2-fluoro-4-phenoxyphenyl)-1H-pyrazolo[3,4-d]pyrimidin-1-yl)methyl)pyrrolidine-1-carbonyl)-4-(2-methoxyethylamino)-4-methylpent-2-enenitrile;
(R)-2-(2-((4-amino-3-(2-fluoro-4-phenoxyphenyl)-1H-pyrazolo[3,4-d]pyrimidin-1-yl)methyl)pyrrolidine-1-carbonyl)-3-(1-aminocyclopropyl)acrylonitrile;
2-[(2S)-2-[[4-amino-3-(2-fluoro-4-phenoxy-phenyl)pyrazolo[3,4-d]pyrimidin-1-yl]methyl]pyrrolidine-1-carbonyl]-4-methyl-4-morpholino-pent-2-enenitrile;
2-[(2R)-2-[[4-amino-3-(2-fluoro-4-phenoxy-phenyl)pyrazolo[3,4-d]pyrimidin-1-yl]methyl]pyrrolidine-1-carbonyl]-4-methyl-4-morpholino-pent-2-enenitrile;
2-[(2R)-2-[[4-amino-3-(2-fluoro-4-phenoxy-phenyl)pyrazolo[3,4-d]pyrimidin-1-yl]methyl]pyrrolidine-1-carbonyl]-4-methyl-4-(1-piperidyl)pent-2-enenitrile;
2-[(2S)-2-[[4-amino-3-(2-fluoro-4-phenoxy-phenyl)pyrazolo[3,4-d]pyrimidin-1-yl]methyl]pyrrolidine-1-carbonyl]-4-methyl-4-(1-piperidyl)pent-2-enenitrile; and
2-[(2S)-2-[[4-amino-3-(2-fluoro-4-phenoxy-phenyl)pyrazolo[3,4-d]pyrimidin-1-yl]methyl]-pyrrolidine-1-carbonyl]-3-(3-methyloxetan-3-yl)prop-2-enenitrile;
or a pharmaceutically acceptable salt of any of the foregoing compounds.

9. The method according to any one of claims 1-5, wherein R^{c} is alkyl.

10. The method according to claim 9, wherein R^{c} is t-butyl.

11. The method according to claim 10, wherein the BTK inhibitor is chosen from (E) isomer, (Z) isomer, and a mixture of (E) and (Z) isomers of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile (Compound (IA)), and/or a pharmaceutically acceptable salt any of the foregoing compounds.

12. The method according to claim 10, wherein the compound is a substantially pure (E) or (Z) isomer of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile (Compound (IA)), and/or a pharmaceutically acceptable salt thereof.

13. The method according to claim 12, wherein at least about 85% w/w of (R)-2-(3-(4-amino-3-(2-fluoro-4-phenoxyphenyl)-1H-pyrazolo[3,4-d]pyrimidin-1-yl)piperidine-1-carbonyl)-4,4-dimethylpent-2-enenitrile or at least about 85% w/w of a pharmaceutically acceptable salt of (R)-2-(3-(4-amino-3-(2-fluoro-4-phenoxyphenyl)-H-pyrazolo[3,4-d]pyrimidin-1-yl)piperidine-1-carbonyl)-4,4-dimethylpent-2-enenitrile is the (E) isomer.

14. The method according to claim 5, wherein R^{c} is substituted alkyl.

15. The method according to claim 14, wherein:
R^{c} is —C(CH₃)₂-(4-R⁸-piperazin-1-yl);
R⁸ is chosen from hydrogen, alkyl, alkoxyalkyl, haloalkyl, alkylsulfonyl, alkoxycarbonyl, acyl, and oxetan-3-yl; and
the piperazinyl ring is additionally optionally and independently substituted with one or two alkyls.

16. The method according to claim 15, wherein the BTK inhibitor is chosen from (E) isomer, (Z) isomer, and a mixture of (E) and (Z) isomers of:
2-[[(3R)-3-[4-amino-3-(2-fluoro-4-phenoxyphenyl)-1H-pyrazolo[3,4-d]-pyrimidin-1-yl]-piperidin-1-yl]carbonyl]-4-methyl-4-(4-methylpiperazin-1-yl)pent-2-enenitrile;
2-((R)-3-(4-amino-3-(2-fluoro-4-phenoxyphenyl)-1H-pyrazolo[3,4-d]pyrimidin-1-yl)piperidine-1-carbonyl)-4-methyl-4-(piperazin-1-yl)pent-2-enenitrile;
2-[(3R)-3-[4-amino-3-(2-fluoro-4-phenoxy-phenyl)pyrazolo[3,4-d]-pyrimidin-1-yl]piperidine-1-carbonyl]-4-(4-ethylpiperazin-1-yl)-4-methyl-pent-2-enenitrile;
(R)-2-(3-(4-amino-3-(2-fluoro-4-phenoxyphenyl)-1H-pyrazolo[3,4-d]pyrimidin-1-yl)piperidine-1-carbonyl)-4-(4-isopropylpiperazin-1-yl)-4-methylpent-2-enenitrile;
(R)-2-(3-(4-amino-3-(2-fluoro-4-phenoxyphenyl)-1H-pyrazolo[3,4-d]pyrimidin-1-yl)piperidine-1-carbonyl)-4-(4-(tert-butyl)piperazin-1-yl)-4-methylpent-2-enenitrile;
(R)-2-(3-(4-amino-3-(2-fluoro-4-phenoxyphenyl)-1H-pyrazolo[3,4-d]pyrimidin-1-yl)piperidine-1-carbonyl)-4-(4-(2-methoxyethyl)piperazin-1-yl)-4-methylpent-2-enenitrile;
(R)-2-(3-(4-amino-3-(2-fluoro-4-phenoxyphenyl)-1H-pyrazolo[3,4-d]pyrimidin-1-yl)piperidine-1-carbonyl)-4-methyl-4-(4-(methylsulfonyl)piperazin-1-yl)pent-2-enenitrile;
(R)-2-(3-(4-amino-3-(2-fluoro-4-phenoxyphenyl)-1H-pyrazolo[3,4-d]pyrimidin-1-yl)piperidine-1-carbonyl)-4-methyl-4-(4-(2,2,2-trifluoroethyl)piperazin-1-yl)pent-2-enenitrile;
2-((R)-3-(4-amino-3-(2-fluoro-4-phenoxyphenyl)-1H-pyrazolo[3,4-d]pyrimidin-1-yl)piperidine-1-carbonyl)-4-methyl-4-((3S,5R)-3,4,5-trimethylpiperazin-1-yl)pent-2-enenitrile;
2-((R)-3-(4-amino-3-(2-fluoro-4-phenoxyphenyl)-1H-pyrazolo[3,4-d]pyrimidin-1-yl)piperidine-1-carbonyl)-4-((3S,5R)-3,5-dimethylpiperazin-1-yl)-4-methylpent-2-enenitrile;
(R)-2-(3-(4-amino-3-(2-fluoro-4-phenoxyphenyl)-1H-pyrazolo[3,4-d]pyrimidin-1-yl)piperidine-1-carbonyl)-4-methyl-4-(4-(oxetan-3-yl)piperazin-1-yl)pent-2-enenitrile;
(R)-4-(4-acetylpiperazin-1-yl)-2-(3-(4-amino-3-(2-fluoro-4-phenoxyphenyl)-1H-pyrazolo[3,4-d]pyrimidin-1-yl)piperidine-1-carbonyl)-4-methylpent-2-enenitrile; and
(R)-methyl-4-(5-(3-(4-amino-3-(2-fluoro-4-phenoxyphenyl)-1H-pyrazolo[3,4-d]pyrimidin-1-yl)piperidin-1-yl)-4-cyano-2-methyl-5-oxopent-3-en-2-yl)piperazine-1-carboxylate;
or a pharmaceutically acceptable salt of any of the foregoing compounds.

17. The method according to claim 15, wherein R⁸ is oxetan-3-yl.

18. The method according to claim 17, wherein the BTK inhibitor is chosen from (E) isomer, (Z) isomer, and a mixture of (E) and (Z) isomers of 2-[(3R)-3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (Compound (IB)); and/or a pharmaceutically acceptable salt of any of the foregoing compounds.

19. The method according to claim 17, wherein the BTK inhibitor is a substantially pure (E) or (Z) isomer of 2-[(3R)-3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile (Compound (IB)), or a pharmaceutically acceptable salt thereof.

20. The method according to claim 19, wherein at least about 85% w/w of 2-[(3R)-3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile or at least about 85% w/w of a pharmaceutically acceptable salt of 2-[(3R)-3-[4-amino-3-(2-fluoro-4-phenoxy-phenyl)pyrazolo[3,4-d]pyrimidin-1-yl]piperidine-1-carbonyl]-4-methyl-4-[4-(oxetan-3-yl)piperazin-1-yl]pent-2-enenitrile is the (E) isomer.

21. The method according to any one of claims 1-20, wherein the pharmaceutical composition is administered in place of a corticosteroid therapy.

22. The method according to any one of claims 1-20, wherein the pharmaceutical composition is administered in combination with a corticosteroid therapy.

23. The method according to any one of claims 1-20, wherein the pharmaceutical composition is administered in combination with a noncorticosteroidal immunosuppressive and/or anti-inflammatory agent.

24. The method according to any one of claims 1-20, wherein the pharmaceutical composition is administered in combination with a corticosteroid maintenance therapy.

25. The method according to any one of claims 1-24, wherein the mammal is a human.

26. The method according to any one of claims 1-25, wherein the pharmaceutical composition is administered in combination with an active pharmaceutical ingredient chosen from interferon alpha, interferon gamma, cyclophosphamide, tacrolimus, mycophenolate mofetil, methotrexate, dapsone, sulfasalazine, azathioprine, an anti-CD20 agent, an anti-TN alpha agent, an anti-IL6 agent toward ligand or its receptors, an anti-IL17 agent to ligand or its receptors, an anti-IL1 agent to ligand or its receptors, an anti-IL2 agent to ligand or its receptors, an anti-CD2 agent, an anti-CD3 agent, an anti-CD80/86 agent, an anti-sphingosine-1-phosphate receptor agent, an anti-C5 agent, an anti-mTOR agent, an anti-calcineurin agent, an anti-BAFF/BlyS agent, leflunomide, and teriflunomide.

27. The method according to any one of claims 1-26, wherein the pharmaceutical composition is administered in combination with rituximab, ofatumumab, obinutuzumab, or veltuzumab, or a biosimilar version of any of the foregoing.

28. The method according to any one of claims 1-26, wherein the pharmaceutical composition is administered in combination with at least one antiviral agent.

29. The method according to claim 28, wherein the antiviral agent comprises at least one agent chosen from entry inhibitors, uncoating inhibitors, reverse transcriptase inhibitors, integrase inhibitors, transcription inhibitors, and protease inhibitors.

30. The method according to claim 28 or 29, wherein the antiviral agent comprises remdesivir.
